(19) United States
(12) Patent Application Publication
(10) Pub. No.: US 2025/0073000 A1
(43) Pub. Date: Mar. 6, 2025

(54) TREATMENT PLANNING USING PATIENT FACIAL IMAGES

(71) Applicant: Align Technology, Inc., San Jose, CA (US)

(72) Inventors: Olga Matusevich, Moscow (RU); Vladislav Andreevich Miryaha, Moscow (RU); Yury A. Brailov, Moscow (RU); Alexander Kulinich, Nizhegorodskaya obl. (RU); Sergey Borisovich Surov, Moscow (RU); Elena Agilina, Holmdel, NJ (US); Boris Aleksandrovich Vysokanov, Moscow (RU); Dmitry Yurievich Chekh, Moscow (RU); Polina Antyasova, Yerevan (AM)

(21) Appl. No.: 18/821,687

(22) Filed: Aug. 30, 2024

Related U.S. Application Data
(60) Provisional application No. 63/579,865, filed on Aug. 31, 2023.

(51) Int. Cl. A61C 7/00; A61C 13/34; G06T 7/00; G16H 20/40

(52) U.S. Cl. CPC A61C 7/002; A61C 13/34; G06T 7/0014; G16H 20/40; G06T 2207/30036

(57) ABSTRACT

Systems and methods for treatment planning using patient facial images are provided. In some embodiments, a method includes receiving a 2D image of a patient's face, generating at least one smile line based on the 2D image, receiving a 3D digital representation of the patient's arch, determining a correspondence between the 2D image and the 3D digital representation, generating a 3D projection of the at least one smile line based on the correspondence, and determining a target arrangement for the patient's teeth based on the 3D projection of the at least one smile line.

### CROSS-REFERENCE TO RELATED APPLICATION(S)

The present application claims the benefit of priority to U.S. Provisional Application No. 63/579,865, filed Aug. 31, 2023, the disclosure of which is incorporated by reference herein in its entirety.

### TECHNICAL FIELD

The present technology generally relates to treatment planning, and in particular, to treatment planning using patient facial images.

### BACKGROUND

Orthodontic and restorative treatment procedures are used to treat dental conditions such as malocclusions, damaged or missing teeth, and jaw dysfunction/misalignment. However, conventional treatment planning software may rely solely on the clinical goals provided by the clinician to determine the appropriate treatment for the patient's teeth, and may lack the ability to consider the aesthetics of the teeth in context with the rest of the patient's face. In some instances, a treatment plan that achieves the clinical goals may nevertheless produce an aesthetically undesirable outcome. Although the output of the treatment planning software may be manually revised to comply with aesthetic preferences, this process is time-consuming, subjective, may produce inconsistent results, and may conflict with clinical constraints and requirements.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic block diagram illustrating a system for treatment planning, in accordance with embodiments of the present technology.

FIG. 2 is a flow diagram providing a general overview of a method for generating a treatment plan for a patient's teeth, in accordance with embodiments of the present technology.

FIG. 3 is a representative example of a patient image annotated with a plurality of smile lines, in accordance with embodiments of the present technology.

FIG. 4A illustrates a target arrangement of a patient's teeth generated by an automated treatment planning algorithm without considering smile lines.

FIG. 4B illustrates a target arrangement of a patient's teeth generated by an automated treatment planning algorithm that takes smile lines into account, in accordance with embodiments of the present technology.

FIG. 5 is a flow diagram illustrating a method for planning a treatment for a patient's teeth based on smile lines, in accordance with embodiments of the present technology.

FIG. 6A illustrates a 2D image of a patient's teeth, in accordance with embodiments of the present technology.

FIG. 6B illustrates a 3D model of a patient's teeth, in accordance with embodiments of the present technology.

FIG. 6C illustrates a 3D model of a patient's tooth and a smile plane, in accordance with embodiments of the present technology.

FIG. 7A illustrates a patient smile with excess visible gingiva.

FIG. 7B illustrates a patient smile with reduced visible gingiva, in accordance with embodiments of the present technology.

FIG. 8 is a flow diagram illustrating a method for planning a treatment for a patient's teeth based on visible gingiva, in accordance with embodiments of the present technology.

FIG. 9 is a representative example of a patient image annotated with a lip contour and a tooth contour, in accordance with embodiments of the present technology.

FIG. 10 is a flow diagram illustrating a method for determining a target arrangement of a patient's teeth to reduce visible gingiva, in accordance with embodiments of the present technology.

FIG. 11A is a schematic illustration of a patient's teeth in a target arrangement corresponding to a generic leveling surface, in accordance with embodiments of the present technology.

FIG. 11B is a schematic illustration of a patient's teeth in a target arrangement corresponding to an adjusted leveling surface, in accordance with embodiments of the present technology.

FIG. 12 is a flow diagram illustrating a workflow for generating a treatment plan to reduce visible gingiva, in accordance with embodiments of the present technology.

FIG. 13A illustrates a representative example of a tooth repositioning appliance configured in accordance with embodiments of the present technology.

FIG. 13B illustrates a tooth repositioning system including a plurality of appliances, in accordance with embodiments of the present technology.

FIG. 13C illustrates a method of orthodontic treatment using a plurality of appliances, in accordance with embodiments of the present technology.

FIG. 14 illustrates a method for designing an orthodontic appliance, in accordance with embodiments of the present technology.

FIG. 15 illustrates a method for digitally planning an orthodontic treatment and/or design or fabrication of an appliance, in accordance with embodiments of the present technology.

### DETAILED DESCRIPTION

The present technology relates to treatment planning based on images of a patient's face. In some embodiments, for example, a method for planning a treatment for a patient's teeth includes receiving a 2D image of the patient's face, such as a photograph showing the patient smiling. The method can include generating at least one smile line based on the 2D image that represents a target smile for the patient. The method can include receiving a 3D digital representation of the patient's arch in an initial arrangement. The method can continue with determining a correspondence between the 2D image and the 3D digital representation, and generating a 3D projection of the at least one smile line based on the correspondence. A target arrangement for the patient's teeth can be determined based on the 3D projection of the at least one smile line. For instance, the positions of one or more teeth can be adjusted to conform more closely to the target smile represented by the smile lines.

As another example, a method for planning a treatment for a patient's teeth can include receiving a 2D image of a patient's face, such as a photograph showing the patient smiling. The method can include determining an amount of visible gingiva in the 2D image, such as by generating contours of the patient's teeth and lip, and measuring the distance of the exposed gingiva between the teeth and lip. A target arrangement for the patient's teeth can be determined based on the amount of visible gingiva. For instance, the target arrangement can maintain or reduce the amount of gingiva that is visible when the patient smiles.

The present technology can provide many advantages compared to conventional treatment planning techniques. For instance, the methods herein can be implemented by automated software algorithms to ensure that aesthetic principles are automatically considered when planning the target arrangement of the patient's teeth, thus saving time by reducing the need for multiple rounds of manual adjustments to revise the treatment plan. Moreover, this approach can improve patient satisfaction with treatment by providing more consistent and aesthetically pleasing outcomes.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” “lower,” “left,” “right,” etc., can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Systems and Methods for Treatment Planning

The present technology provides systems and methods for planning a treatment for a patient's dentition. In some embodiments, the treatment is or includes an orthodontic treatment procedure. An orthodontic treatment procedure can involve applying a series of dental appliances (e.g., aligners, palatal expanders) that are configured to incrementally move the teeth through a series of intermediate tooth arrangements. Some or all of the dental appliances can include a polymeric shell including a plurality of teeth-receiving cavities configured to receive and resiliently reposition the teeth toward a particular intermediate tooth arrangement. Additional details and examples of dental appliances suitable for use with the present technology are provided in Section II below.

Alternatively or in combination, the treatment can be or include a restorative treatment procedure. A restorative treatment procedure can involve applying at least one restorative object to the patient's arch to increase a mass of an existing tooth or replace a missing tooth (“tooth mass addition”), removing a portion of an existing tooth (“tooth mass reduction”), or suitable combinations thereof. Examples of restorative objects (also referred to herein as “restoratives” or “restorations”) include, but are not limited to, crowns, veneers, edge bonding, composites, implants, and prosthetics. In some instances, to aid the fitting of the restorative object over an existing tooth, a portion of the tooth can be removed to provide a mounting surface to receive the restorative object. Different types of restorative objects may require differing amounts of tooth reduction (e.g., a veneer may require less tooth mass reduction than a crown). The amount of tooth mass reduction can also vary depending on the position of the tooth. Optionally, one or more neighboring teeth may also undergo tooth mass reduction to provide space for the restorative object.

In some embodiments, the treatment is a combined orthodontic and restorative treatment procedure, also known as an “orthodontic-restorative” or “ortho-restorative” treatment procedure. An ortho-restorative treatment procedure can include: (1) an orthodontic treatment procedure in which one or more teeth are repositioned from an initial tooth arrangement toward a target tooth arrangement, and (2) a restorative treatment procedure in which the shape of one or more existing teeth is modified and/or one or more missing teeth are replaced. The orthodontic and restorative treatment procedures can be performed sequentially (e.g., all tooth repositioning is performed before any restorative treatments are performed, or vice-versa), concurrently (e.g., any particular stage of the treatment plan can include both tooth repositioning and restorative treatment), or suitable combinations thereof.

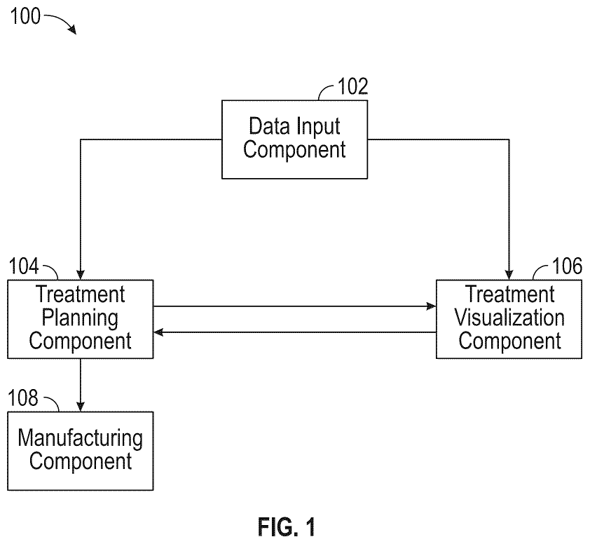

FIG. 1 is a schematic block diagram illustrating a system 100 for treatment planning, in accordance with embodiments of the present technology. The system 100 can be configured to provide a software platform that provides a single ecosystem for planning and visualizing orthodontic treatment procedures, restorative treatment procedures, ortho-restorative treatment procedures, and/or other treatment procedures performed on the patient's craniofacial region. The system 100 includes a data input component 102, a treatment planning component 104, a treatment visualization component 106, and a manufacturing component 108.

The data input component 102 is configured to receive patient data from one or more input devices. The patient data can include any data relevant to a treatment procedure for the patient. For example, the patient data can include data of the patient's teeth, gingiva, arch, intraoral cavity, jaws, face, and/or any other hard or soft tissues of the craniofacial region. The patient data can include photographs, videos, scan data (e.g., intraoral and/or extraoral scans), magnetic resonance imaging (MRI) data, radiographic data (e.g., standard x-ray data such as bitewing x-ray data, panoramic x-ray data, cephalometric x-ray data, computed tomography (CT) data, cone-beam computed tomography (CBCT) data, fluoroscopy data), motion data, and the like. The patient data can include 2D data (e.g., 2D photographs or videos), 3D data (e.g., 3D photographs, intraoral and/or extraoral scans, digital models), 4D data (e.g., fluoroscopy data, dynamic articulation data, hard and/or soft tissue motion capture data), or suitable combinations thereof.

The data input component 102 can be operably coupled to various peripheral devices (not shown) in order to receive patient data therefrom. The peripheral devices can be associated with and/or operated by a healthcare provider (e.g., a clinician), a technician, a patient, or any other suitable user. The peripheral devices can be or include a computing device (e.g., personal computer, laptop, workstation, server, mobile device) that receives, stores, and/or processes the patient data for transmission to the data input component 102. The patient data can be transmitted to the data input component 102 via any suitable combination of wired and/or wireless communication methods.

In some embodiments, for example, the data input component 102 receives data from a scanner configured to obtain a 3D digital representation (e.g., images, surface topography data) of a patient's teeth, such as via direct intraoral scanning or indirectly via casts, impressions, models, etc. The scanner can include a probe (e.g., a handheld probe) for optically capturing 3D structures (e.g., by confocal focusing of an array of light beams). Examples of scanners suitable for use with the system 100 include, but are not limited to, the iTero® intraoral digital scanner manufactured by Align Technology, Inc., the 3M True Definition Scanner, and the Cerec Omnicam manufactured by Sirona®. The data obtained by the scanner can be transmitted to a clinician's computing device, which in turn can transmit the data to the data input component 102.

As another example, the data input component 102 can receive patient data from an imaging device (e.g., a camera) that generates image data of a patient's teeth, arch, jaws, face, head, and/or other craniofacial anatomy. The image data can include one or more 2D images (e.g., photographs) that depict the patient from one or more views, such as a profile view of the patient's head, a front view of the patient's head with a neutral expression, a front view of the patient's head while smiling, a view of the upper jaw, a view of the lower jaw, a right buccal view with the jaw closed, an anterior view with the jaw closed, a left buccal view with the jaw closed, a right buccal view with the jaw open, an anterior view with jaw open, and/or a left buccal view with the jaw open. Alternatively or in combination, the image data can include video data of the patient, such as video data showing the patient smiling, speaking, moving their jaws, turning their head, etc. In some embodiments, the imaging device is part of or is operably coupled to a mobile device (e.g., smartphone, tablet), which can be operated by the patient, by a healthcare provider (e.g., a clinician), or other suitable user. The mobile device can implement a mobile application that instructs the user to capture the image data, then transmits the image data to the data input component 102.

The treatment planning component 104 is configured to generate a treatment plan for the patient, based on the patient data from the data input component 102. As previously discussed, the treatment plan can include an orthodontic treatment, a restorative treatment, or a combined ortho-restorative treatment. In some embodiments, for example, the treatment planning component 104 is configured to receive a digital representation of an initial tooth arrangement of a patient from the data input component 102. The treatment planning component 104 can then determine a target tooth arrangement to be achieved via orthodontic and/or restorative treatment. The target tooth arrangement can be an arrangement of the patient's teeth that achieves a desired aesthetic and/or functional treatment goal (e.g., correct malocclusions and/or repair missing, malformed, and/or damaged teeth). The target tooth arrangement can be determined based at least in part on image data of the patient, such as a photograph of the patient's smile. The treatment planning component 104 can then generate a treatment plan for achieving the target tooth arrangement, such as a series of intermediate tooth arrangements configured to reposition the teeth from the initial tooth arrangement toward the target tooth arrangement and/or one or more tooth mass modifications. The target tooth arrangement and treatment plan can be determined manually based on input from a technician, automatically using software algorithms, or suitable combinations thereof. Additional details of the processes that can be performed by the treatment planning component 104 are described further below.

The treatment visualization component 106 is configured to output a visualization that graphically represents the treatment plan generated by the treatment planning component 104. For example, in embodiments where the treatment plan includes repositioning the patient's teeth from the initial tooth arrangement toward the target tooth arrangement via a series of intermediate tooth arrangements, the treatment visualization component 106 can output a plurality of 3D models and/or 2D images showing the initial tooth arrangement, target tooth arrangement, and/or intermediate tooth arrangements. As another example, in embodiments where the treatment plan includes tooth mass addition and/or reduction, the treatment visualization component 106 can show the amounts and/or locations of tooth mass addition and/or reduction. Optionally, the treatment visualization component 106 can also receive and display patient data received from the data input component 102 (e.g., an image of the patient's smile) to provide additional guidance to a user reviewing the treatment plan. In some embodiments, the treatment visualization component 106 displays multiple types of patient data (e.g., 2D, 3D, and/or 4D data) concurrently using graphical user interface elements such as side-by-side views, overlays (e.g., in which each layer can be independently turned on, turned off, or adjusted in opacity), animations, etc. This approach allows the user to visualize the planned treatment in different contexts, e.g., with respect to the patient's facial features, soft tissues, hard tissues, jaw articulation, etc. Additional details of the processes that can be performed by the treatment visualization component 106 are described further below.

In some embodiments, the treatment plan produced by the treatment planning component 104 is displayed to a user (e.g., clinician, technician, patient) via the visualization produced by the treatment visualization component 106. The treatment visualization component 106 can also provide user interface tools allowing the user to provide feedback on the treatment plan, as described in detail below. For example, the user can modify the treatment plan, such as by changing the positions of one or more teeth, changing an amount of tooth mass addition and/or reduction, changing the shape of a restorative object, changing the number of treatment stages, etc. The feedback can be used to directly update the treatment plan, or can be transmitted to the treatment planning component 104, which can update the treatment plan accordingly. The updated treatment plan can be transmitted back to the treatment visualization component 106 for further user review. This process can be repeated until the user approves the treatment plan.

Optionally, once the treatment plan is approved, the treatment planning component 104 can transmit instructions (e.g., STL files, CLI files, CAD files) to the manufacturing component 108 for fabricating one or more devices for use with the treatment plan. For example, the manufacturing component 108 can produce a series of dental appliances (e.g., aligners, palatal expanders) configured to reposition the patient's teeth from the initial tooth arrangement toward the target tooth arrangement. The manufacturing component 108 can also produce attachments, attachment placement templates, and/or other devices to be used in conjunction with a dental appliance, e.g., to improve control over the forces on the patient's teeth. As another example, the manufacturing component 108 can produce one or more restorative objects to be applied to the patient's arch, such as a crown, veneer, prosthetic, implant, etc. In a further example, the manufacturing component 108 can produce a guide or template to be placed in the patient's intraoral cavity to assist a clinician in performing a treatment procedure, such as preparing a tooth for a restorative object, performing tooth mass addition or reduction, placing an attachment or restorative object on a tooth, etc.

In some embodiments, the manufacturing component 108 is configured to fabricate the device(s) using an additive manufacturing technique. Additive manufacturing (also referred to herein as “3D printing”) includes a variety of technologies which fabricate 3D objects directly from digital models through an additive process. In some embodiments, additive manufacturing includes depositing a precursor material (e.g., a polymerizable resin) onto a build platform. The precursor material can be cured, polymerized, melted, sintered, fused, and/or otherwise solidified to form a portion of the object and/or to combine the portion with previously formed portions of the object. In some embodiments, the additive manufacturing techniques provided herein build up the object geometry in a layer-by-layer fashion, with successive layers being formed in discrete build steps. Alternatively or in combination, the additive manufacturing techniques described herein can allow for continuous build-up of an object geometry.

Examples of additive manufacturing techniques suitable for use with the methods described herein include, but are not limited to, the following: (1) vat photopolymerization, in which an object is constructed from a vat or other bulk source of liquid photopolymer resin, including techniques such as stereolithography (SLA), digital light processing (DLP), continuous liquid interface production (CLIP), two-photon induced photopolymerization (TPIP), and volumetric additive manufacturing (VAM); (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) material extrusion, in which material is drawn though a nozzle, heated, and deposited layer-by-layer, such as fused deposition modeling (FDM) and direct ink writing (DIW); (5) powder bed fusion, including techniques such as direct metal laser sintering (DMLS), electron beam melting (EBM), selective heat sintering (SHS), selective laser melting (SLM), and selective laser sintering (SLS); (6) sheet lamination, including techniques such as laminated object manufacturing (LOM) and ultrasonic additive manufacturing (UAM); and (7) directed energy deposition, including techniques such as laser engineering net shaping, directed light fabrication, direct metal deposition, and 3D laser cladding. Optionally, an additive manufacturing process can use a combination of two or more additive manufacturing techniques.

In some embodiments, the system 100 is used to monitor and/or update a treatment plan after the treatment procedure has already started. For example, the data input component 102 can receive patient data indicative of a state of the patient's teeth, gingiva, arch, jaws, face, etc., after the start of treatment. The patient data can be transmitted to the treatment planning component 104 for comparison with the original treatment plan. If the treatment planning component 104 determines that the treatment plan should be modified (e.g., the patient's teeth are off-course), the treatment planning component 104 can generate a revised treatment plan. For example, the revised treatment plan can include modifications to the target tooth arrangement and/or to one or more intermediate tooth arrangements. The revised treatment plan can be transmitted to the treatment visualization component 106 for user review. Once the revised treatment plan is approved, the treatment planning component 104 can send instructions to the manufacturing component 108 to fabricate one or more devices for implementing the revised treatment plan (e.g., new dental appliances, attachments, restorative objects, etc.). This process can be repeated as desired until the patient has achieved the desired treatment goal.

The system 100 illustrated in FIG. 1 can be configured in many different ways. For example, the various components 102-108 of the system 100 can be implemented by one or more computing devices (e.g., a server, personal computer, workstation, mainframe, laptop, mobile device) having software and hardware components (e.g., processors, memory, user input and output devices, network interfaces, etc.) configured to perform the various operations described herein. For example, some or all of the components 102-108 can be implemented as a distributed “cloud” service across any suitable combination of hardware and/or virtual computing resources. In some embodiments, some or all of the components 102-108 can be disposed on a single computing device and/or can be part of a single communications network. Alternatively, some or all of the components 102-108 can be located on distinct and separate computing devices. The components 102-108 can be operably coupled via one or more communications networks, such as any of the following: a wired network, a wireless network, a metropolitan area network (MAN), a local area network (LAN), a wide area network (WAN), a virtual local area network (VLAN), an internet, an extranet, an intranet, and/or any other suitable type of network or combinations thereof.

Although FIG. 1 illustrates the components 102-108 of the system 100 as being separate functional elements, in other embodiments, some or all of the components 102-108 can be combined. For example, the data input component 102 can be combined with the treatment planning component 104, the treatment planning component 104 can be combined with the treatment visualization component 106, etc. Additionally, any of the components 102-108 can be divided into smaller sub-components, and/or the system 100 can include other components not shown in FIG. 1.

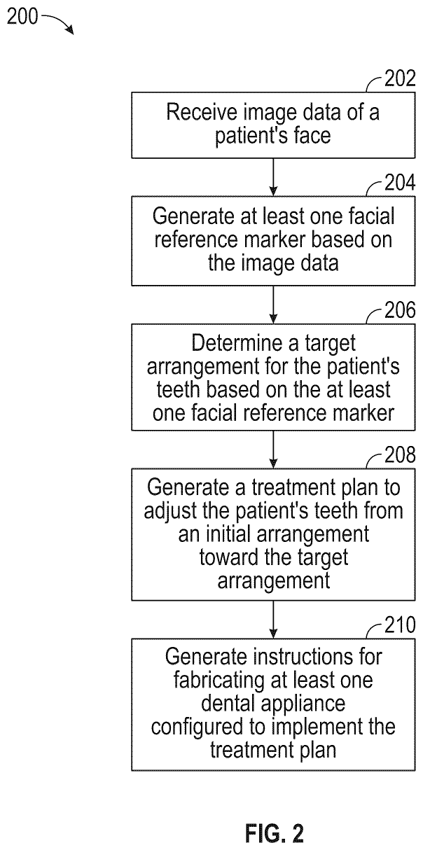

FIG. 2 is a flow diagram providing a general overview of a method 200 for generating a treatment plan for a patient's teeth, in accordance with embodiments of the present technology. The method 200 can be performed by any suitable system or device, such as the by the data input component 102 and/or treatment planning component 104 of the system 100 of FIG. 1. In some embodiment, some or all of the processes of the method 200 are implemented as computer-readable instructions (e.g., program code) that are configured to be executed by one or more processors of a computing device.

The method 200 can begin at block 202 with receiving image data of a patient's face. The image data can be received from any suitable imaging device, such as a camera. The imaging device can be a standalone device (e.g., DSLR camera) or can be integrated into another device (e.g., the camera of a mobile device). The image data can include the patient's mouth region, including the visible portions of the teeth and gingiva, as well as the patient's lips. The image data can include at least one image of the patient's mouth in one or more positions. For example, the patient's mouth can be in a smiling position (e.g., a social smiling position), a repose position with relaxed muscles and lips slightly parted, an anterior retracted open bite or closed bite position, etc. Optionally, the image data can also depict other parts of the patient's anatomy, such as other facial features (e.g., eyes, eyebrows, nose, subnasion, checks, chin, jawline), head, neck, shoulders, and/or torso, or the entire body of the patient. For instance, the image data can include at least one image showing the patient's entire face from a frontal view while the patient is smiling (“full-face smile image”). As another example, the image data can include at least one image showing the patient's entire face from a frontal view while the patient's mouth is in a repose position (“full-face repose image”). In a further example, the image data can include at least one intraoral image (e.g., an intraoral anterior image and/or intraoral buccal image). In some embodiments, the image data includes a single 2D image, such as a single photograph of the patient smiling. In other embodiments, the image data can include multiple 2D images, such as multiple photographs showing different views of the patient's face or a sequence of image frames of a video.

At block 204, the method 200 can continue with generating at least one facial reference marker based on the image data. The facial reference marker(s) can include one or more points, lines, curves, contours, shapes, and/or any other geometric element corresponding to the unique anatomical features of the patient's face. At least some of the facial reference markers can indicate one or more characteristics of a facial feature, such as the location, size (e.g., height, width, length), and/or shape (e.g., curvature, proportion) of the facial feature. Optionally, some of the facial reference markers can indicate spatial relationships between facial features, such as the distance between a first facial feature and a second facial feature (e.g., distance between the canines, distance between the lower edge of the upper lip and the gingival margins of the upper teeth). Some facial markers can correspond to the actual features of the patient's face (e.g., the location of the facial midline can correspond to the actual line of symmetry of the patient's face, the lip contours can correspond to the actual locations and curvature of the patient's lips), while other facial markers can define a target location and/or shape for a facial feature (e.g., target positions of the visible teeth, target curvature of the patient's smile).

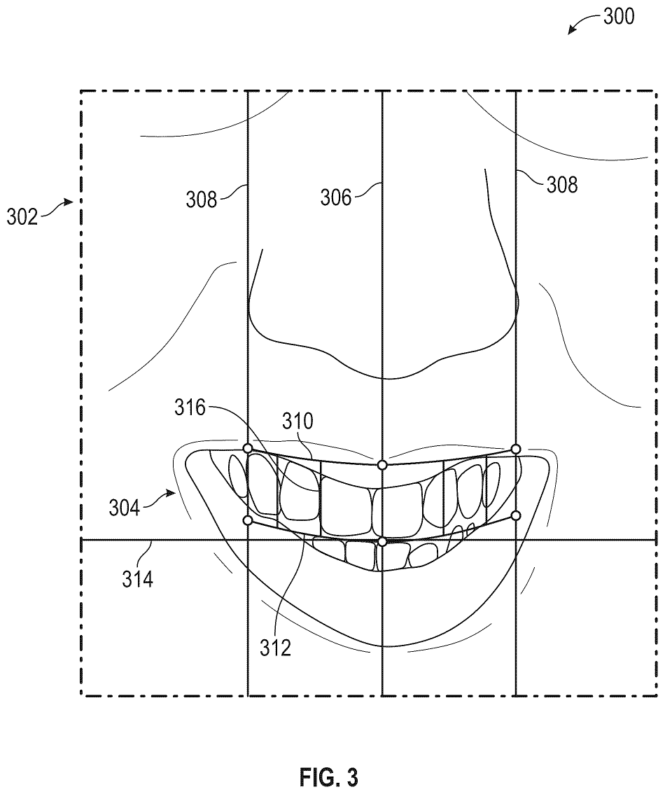

For example, the facial reference marker(s) can include one or more smile lines that define a target smile for a patient (e.g., a smile that is considered aesthetically pleasing based on the patient's particular facial anatomy). The smile lines can define one or more parameters of a target smile, such as the curvature of the smile (e.g., curvature of the incisal edges and/or gingival margins of the teeth of the upper arch), the midline of the smile, and/or the locations and/or geometry of the teeth exposed by the smile (e.g., central incisors, lateral incisors, and/or canines of the upper arch). Additional details and examples of smile lines are provided below in connection with FIG. 3.

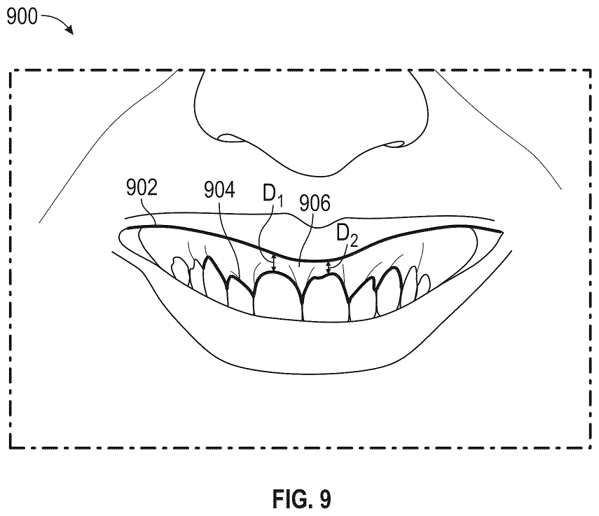

As another example, the facial reference marker(s) can include one or more geometric elements within the mouth region that can be used to determine an amount of visible gingiva in the patient's smile. For example, the facial reference marker(s) can include a lip contour (also known as a “lip curve” or “lip spline”) corresponding to the lower edge of the patient's upper lip, and a tooth contour corresponding to the gingival margins of the visible teeth of the patient's upper arch. The distance between the lip contour and tooth contour can be used to evaluate the amounts and locations of gingiva of the upper arch that is exposed when the patient smiles. Additional details and examples of facial reference markers for assessing visible gingiva are provided below in connection with FIG. 9.

In some embodiments, the facial reference marker(s) are generated by a software platform for treatment planning, such as the treatment planning component 104 of the system 100 of FIG. 1. The software platform can implement an automated facial reference marker generation algorithm that utilizes suitable computer vision and/or machine learning techniques (e.g., convolutional neural networks (CNNs) and/or other deep learning techniques) to analyze the image data to identify the locations of one or more facial reference markers. Optionally, the facial reference marker(s) generated by the algorithm can be displayed to a user (e.g., a technician or clinician) for review. The user can view a digital representation of the facial markers (e.g., overlaid on the image data of the patient), e.g., via the treatment visualization component 106 of the system 100 of FIG. 1, and can approve or make modifications to the facial reference marker(s) as desired.

At block 206, the method 200 can include determining a target arrangement for the patient's teeth based on the at least one facial reference marker. The target arrangement can be a prescribed arrangement of the teeth that meets the patient's desired aesthetic and/or clinical treatment goals. For example, the target arrangement can correspond to an improved (e.g., “ideal”) arch form and/or smile for the patient. In some embodiments, the process of block 206 involves receiving a digital representation of an initial arrangement of the patient's teeth (e.g., intraoral scan data), analyzing the initial arrangement to identify indications to be treated (e.g., maloccluded, malformed, damaged, and/or missing teeth), then determining a target arrangement that would correct some or all of the indications through orthodontic repositioning, tooth mass modification, or a combination thereof. For instance, tooth repositioning can be prescribed to correct malocclusions and/or to create space for restorative objects to be applied to the patient's arch. Tooth mass modifications can be prescribed for teeth that are damaged, malformed, missing, or otherwise deviate from the desired shape, and/or to create space for orthodontic movements. Accordingly, the target arrangement can include (1) one or more teeth that have been repositioned (e.g., via tipping, translation, rotation, extrusion, intrusion, root movement) relative to the initial tooth arrangement, and/or (2) one or more teeth that have undergone a change in mass (e.g., tooth mass addition, tooth mass reduction) relative to the initial arrangement.

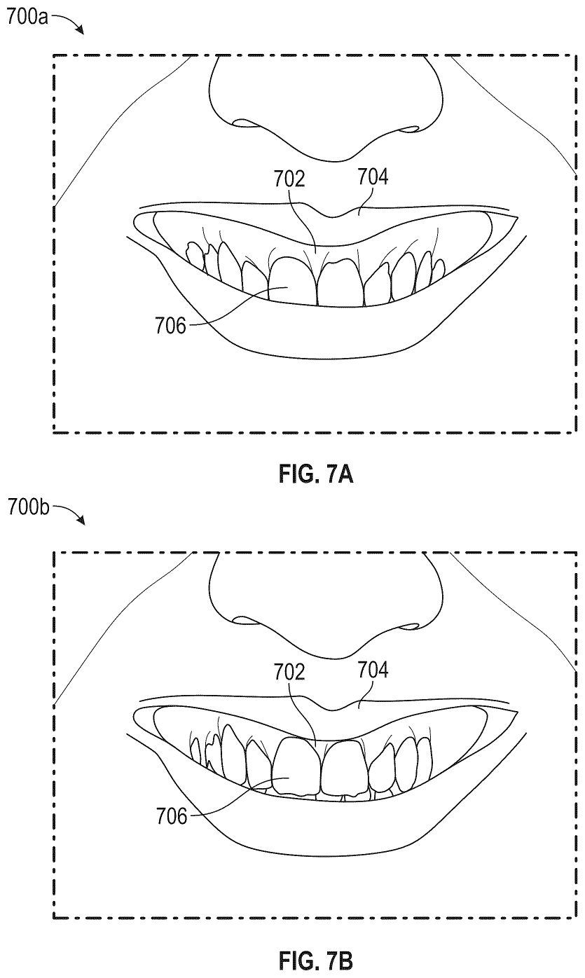

In some embodiments, the target arrangement is designed at least partly based on the patient's unique facial features, also referred to herein as “facially-driven” treatment planning. Facially-driven treatment planning can include, for example, using the facial reference marker(s) generated in block 204 to determine a target smile for the patient, then generating a target tooth arrangement that would produce the target smile. For example, smile lines can be used to determine the target positions and/or shapes of the patient's teeth that would cause the patient's smile to conform more closely to the target smile defined by the smile lines, as described further below in connection with FIGS. 3-6C. Alternatively or in combination, lip and teeth contours can be used to determine the positions of the patient's teeth that would reduce the amount of visible gingiva in the patient's smile, as described further below in connection with FIGS. 7A-12. Optionally, the target arrangement can also be determined based in part on other considerations, such as orthodontic principles, a prescription from a clinician, and/or patient preference.

In some embodiments, the target arrangement is determined by a software platform for treatment planning, such as the treatment planning component 104 of the system 100 of FIG. 1. The software platform can implement an automated treatment planning algorithm that utilizes the facial reference marker(s) in generating the target arrangement. In some embodiments, the treatment planning algorithm is a rule-based algorithm that applies a set of rules to determine the target arrangement, and at least some of the rules are related to the facial reference marker(s). For instance, the algorithm can implement at least one rule that indicates how one or more teeth in the target arrangement should be positioned relative to one or more facial reference markers (e.g., whether a tooth should be aligned with a facial reference marker, intersect the facial reference marker, be spaced apart from the facial reference marker, etc.). As another example, the algorithm can implement at least one rule that sets a target value or range for a parameter associated with one or more facial reference markers (e.g., a maximum amount of visible gingiva in the patient's smile). In yet another example, the algorithm can implement at least one rule that selects a technique for determining the position of one or more teeth, based on one or more facial reference markers. Optionally, different rules can be weighted differently or otherwise prioritized over other rules, e.g., based on considerations such as clinical efficacy, aesthetics, clinician preference, patient preference, etc.

In some embodiments, the target arrangement generated by the treatment planning algorithm is transmitted to a user (e.g., technician or clinician) for review. For instance, the target arrangement can be displayed to the user via the treatment visualization component 106 of the system 100 of FIG. 1. The user can view a digital representation (e.g., 3D digital model) of the target arrangement, and approve or modify the target arrangement as appropriate. Optionally, the user can provide feedback to the treatment planning algorithm for generating a modified version of the target arrangement. The feedback can include adjustments to one or more facial reference markers (e.g., changing the size, shape, location, etc., of a smile line). The treatment planning algorithm can then use the adjusted facial reference markers to produce a modified target arrangement. This process can be repeated until a desired target arrangement is achieved.

At block 208, the method 200 can include generating a treatment plan to adjust the patient's teeth from an initial arrangement toward the target arrangement. For example, the treatment plan can include a series of intermediate tooth arrangements to reposition the patient's teeth from the initial arrangement toward the target arrangement. Each intermediate arrangement can correspond to an orthodontic treatment stage to be achieved with a respective dental appliance. The intermediate arrangements can be generated automatically using a treatment planning algorithm, manually based on input from a technician, or suitable combinations thereof.

At block 210, the method 200 can include generating instructions for fabricating at least one dental appliance configured to implement the treatment plan. As described in greater detail elsewhere herein, the dental appliances can be configured to incrementally reposition the patient's teeth from the initial arrangement toward the target arrangement according to the treatment plan. In some embodiments, the instructions are configured for manufacturing the dental appliance using direct fabrication, e.g., by directly printing the appliance in accordance with the various additive manufacturing techniques described herein. In other embodiments, the instructions can be configured for indirect fabrication of the appliance, e.g., by thermoforming the appliance over a mold of the patient's teeth.

The method 200 can be varied in many ways. For example, some of the processes shown in FIG. 2 can be omitted (e.g., the processes of block 208 and/or block 210), and/or the method 200 can include additional processes not shown in FIG. 2. Moreover, the method 200 can be combined with any of the other methods described herein.

FIGS. 3-6C illustrate the use of smile lines for planning a treatment for a patient's teeth, in accordance with embodiments of the present technology. As described herein, smile lines can be used to define an aesthetic target smile for a patient, and thus can be used to determine adjustments to positions and/or shapes of the patient's teeth so that the patient's smile conforms more closely to the target smile. In some embodiments, the techniques herein allow for automated treatment planning that takes smile lines into account when planning the target arrangement for the patient's teeth to be achieved via orthodontic and/or restorative treatment procedures.

FIG. 3 is a representative example of a patient image 300 annotated with a plurality of smile lines, in accordance with embodiments of the present technology. The patient image 300 can depict at least a portion of the patient's face 302, including a mouth region 304 with the patient's lips and teeth in a smiling position. The smile lines can include a facial midline 306, a pair of intercanine width (ICW) lines 308, a gingival line 310, an incisal edge line 312, a horizontal line 314, and/or a plurality of proportion lines 316. The facial midline 306 can be a vertical line corresponding to the center of the patient's face 302 (e.g., the line of symmetry of the patient's face 302). The ICW lines 308 can be vertical lines indicating the positions of the right and left canines of the upper arch, respectively, such that the distance between the ICW lines 308 corresponds to the ICW of the patient. The gingival line 310 can be a curved line corresponding to the gingival margin of the teeth of the upper arch. The incisal edge line 312 can be a curved line corresponding to the incisal apices of the teeth of the upper arch. The horizontal line 314 can be orthogonal to the facial midline 306 and can be tangential to the lowest point of the gingival line 310. The proportion lines 316 can correspond to proportions of the patient's upper front teeth (e.g., central incisors, lateral incisors, canines), such as the ratio of the height of central incisor to the width of the central incisors (“centrals height to width ratio”), the ratio of the width of the lateral incisors to the width of the central incisors (“laterals to centrals to width ratio”), and/or the ratio of the width of the canine to the width of the lateral incisors (“canines to laterals width ratio”). In some embodiments, the facial midline 306 represents the actual midline (line of symmetry) of the patient's face 302, while the ICW lines 308, gingival line 310, incisal edge line 312, horizontal line 314, and proportion line 316 represent features of the “ideal” target smile for the patient that are determined from the actual features of the patient's face.

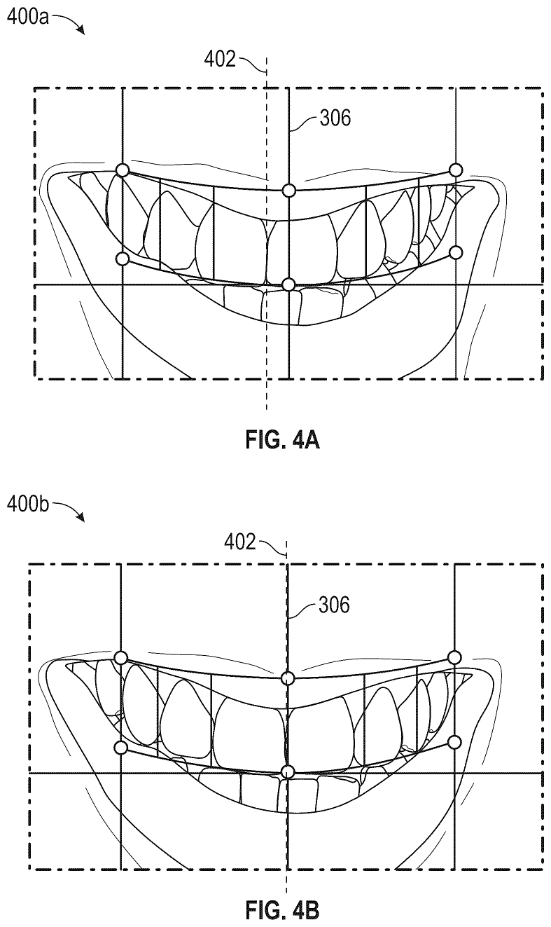

In some instances, treatment planning that does not take smile lines into consideration may produce suboptimal results or may even worsen the positions of the teeth from an aesthetic point of view. For example, FIG. 4A illustrates a target arrangement 400a of a patient's teeth generated by an automated treatment planning algorithm without considering smile lines. As shown in FIG. 4A, the dental midline 402 in the target arrangement 400a is offset from the facial midline 306, which may be aesthetically undesirable due to the resulting asymmetry in the smile. Other issues that may arise include canting (e.g., teeth on one side of the mouth may be vertically misaligned with teeth on other side of the mouth) and improper leveling (e.g., neighboring teeth may be vertically misaligned with each other, posterior teeth may be vertically misaligned with anterior teeth). Such issues may arise because the automated treatment planning algorithm does not have any information regarding the positions of the teeth relative to the rest of the patient's face.

In contrast, treatment planning that incorporates smile lines can produce aesthetically improved results, since the positions of the teeth relative to the patient's other facial features can also be considered. For example, FIG. 4B illustrates a target arrangement 400b of a patient's teeth generated by an automated treatment planning algorithm that takes smile lines into account, in accordance with embodiments of the present technology. As shown in FIG. 4B, the dental midline 402 in the target arrangement 400b is aligned with the facial midline 306, thus producing a more symmetric and aesthetically pleasing smile.

In some embodiments, the automated treatment planning algorithm operates using 3D models of the patient's teeth. For instance, the input to the treatment planning algorithm can be a 3D model of the patient's teeth in an initial arrangement. The output of the treatment planning algorithm can include a 3D model of a target arrangement for the patient's teeth, and, optionally, one or more 3D models of one or more intermediate arrangements to adjust the teeth from the initial arrangement toward the target arrangement. However, the smile lines may be generated from a 2D image of the patient, and thus may be defined relative to a 2D reference frame (e.g., a 2D coordinate system), rather than a 3D reference frame (e.g., a 3D coordinate system). Moreover, the spatial relationship between the 2D reference frame of the smile lines and the 3D reference frame of the 3D models of the teeth may be unknown, such that the smile lines cannot be directly mapped to the 3D models of the teeth. Accordingly, in order to incorporate smile lines into the treatment planning algorithm, the present disclosure provides techniques for projecting the 2D smile lines into the 3D reference frame of the 3D models.

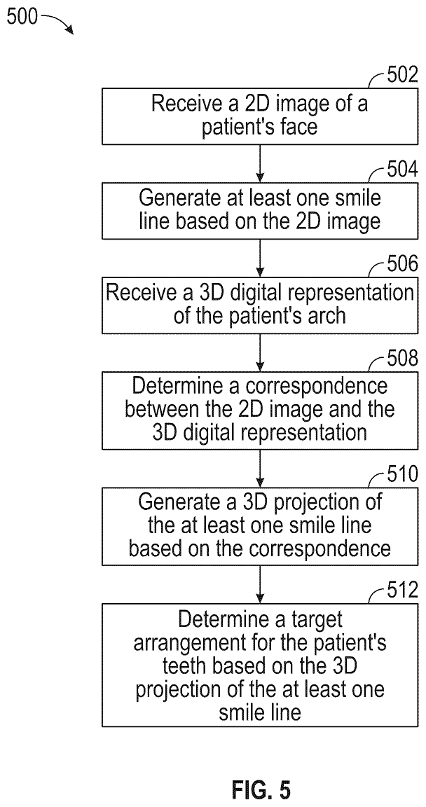

FIG. 5 is a flow diagram illustrating a method 500 for planning a treatment for a patient's teeth based on smile lines, in accordance with embodiments of the present technology. The method 500 can be performed by any suitable system or device, such as the by the data input component 102 and/or treatment planning component 104 of the system 100 of FIG. 1. In some embodiment, some or all of the processes of the method 500 are implemented as computer-readable instructions (e.g., program code) that are configured to be executed by one or more processors of a computing device.

The method 500 can begin at block 502 with receiving a 2D image of a patient's face. The 2D image (e.g., a photograph or an image frame of a video) can be received from any suitable imaging device, such as a camera (e.g., a DSLR camera or a camera of a mobile device). The 2D image can include the patient's mouth region, including the visible portions of the teeth and gingiva, as well as the patient's lips. The patient's mouth can be in any suitable position, such as a smiling position (e.g., a social smiling position). Optionally, the 2D image data can show other parts of the patient's anatomy, such as other facial features (e.g., eyes, eyebrows, nose, subnasion, checks, chin, jawline), head, neck, shoulders, and/or torso, or the entire body of the patient.

At block 504, the method 500 can continue with generating at least one smile line based on the 2D image. The smile line(s) can define one or more parameters of a target smile for the patient, such as the curvature of the smile (e.g., curvature of the incisal edges and/or gingival margins of the teeth of the upper arch), the midline of the smile, and/or the locations and/or geometry of the teeth exposed by the smile (e.g., central incisors, lateral incisors, and/or canines of the upper arch). For instance, the smile line(s) can include any of the smile lines described above in connection with FIG. 3, such as a facial midline, ICW lines, gingival line, incisal edge line, horizontal line, and/or proportion lines.

The smile line(s) can be generated using any suitable technique. For instance, the process of block 504 can include identifying one or more facial landmarks in 2D image, then generating the smile line(s) based on the facial landmarks. The facial landmarks can be reference points corresponding to various anatomical features of the patient's face, such as the eyes, eyebrows, nose, subnasion, mouth, lips, teeth, gingiva, cheeks, chin, and/or jawline. The identification of the facial landmarks can be performed by a facial landmark detection algorithm utilizing suitable computer vision and/or machine learning techniques (e.g., CNNs and/or other deep learning techniques). The input to the algorithm can be the 2D image, and the output of the algorithm can be the locations (e.g., x- and y-coordinates) of each facial landmark in the 2D image.

The smile lines can then be automatically determined based on the identified facial landmark(s). For instance, the facial midline can be determined using facial landmarks associated with symmetric facial features such as the eyes, eyebrows, nose, and/or subnasion. The ICW lines can be determined based on facial landmarks of the patient's eyes. The proportion lines can be determined based on the patient's face type, which can be calculated from facial landmarks of the glabella, chin, cheekbones, and/or jawline. The gingival line, incisal edge line, and/or horizontal line can be determined based on facial landmarks of the patient's teeth, lips, and/or gingiva. Additional details and examples of methods for determining smile lines are provided in U.S. Patent Application Publication No. 2023/0132201 and U.S. Pat. No. 10,758,322, the disclosures of each of which are incorporated by reference herein in their entirety.

At block 506, the method 500 can include receiving a 3D digital representation of the patient's arch. The 3D digital representation can be a surface model, mesh model, parametric model, or any other digital model of the 3D topography of the teeth and gingiva of the patient's arch (e.g., the upper arch of the patient's dentition). As described elsewhere herein, the 3D digital representation can be generated from image data of the patient's arch, such as intraoral scan data. In some embodiments, the 3D digital representation depicts the patient's arch in an initial arrangement before the start of treatment.

At block 508, the method 500 can include determining a correspondence between the 2D image and the 3D digital representation. In some embodiments, the 2D image is provided in a 2D reference frame (e.g., a 2D coordinate space), while the 3D digital representation of the patient's teeth is in a 3D reference frame (e.g., a 3D coordinate space). The process of block 508 can involve projecting the 2D image into the 3D reference frame of the 3D digital representation. Subsequently, the projected 2D image can be used to determine a correspondence between the 2D pixel locations in the 2D image and the corresponding 3D coordinate locations in the 3D reference frame. The correspondence can be a mapping, transformation, function, etc., that can be applied to a 2D pixel location to determine the corresponding 3D location.

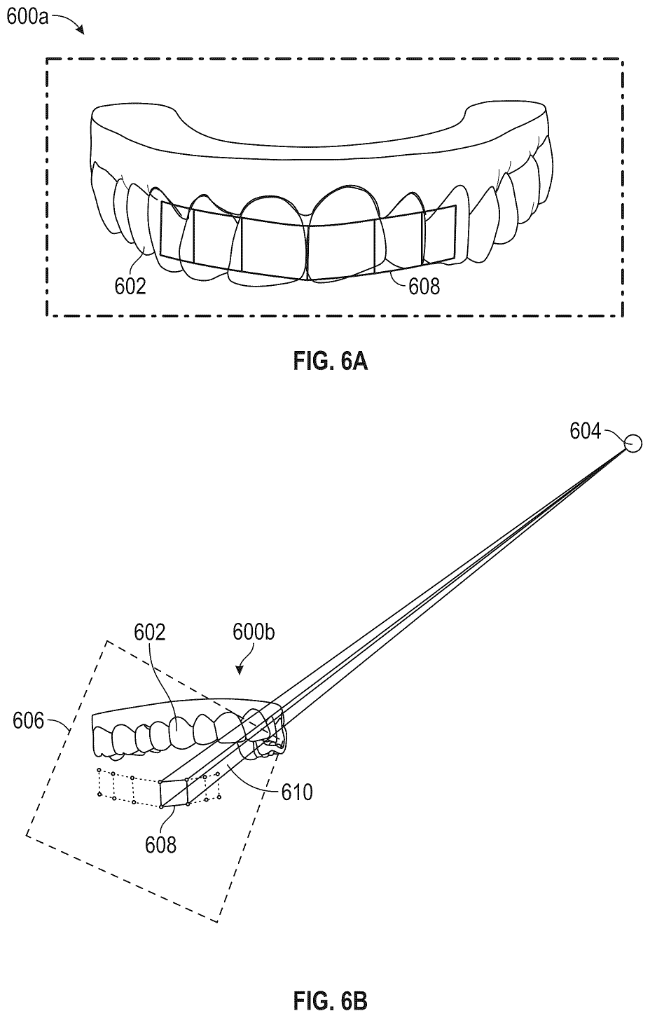

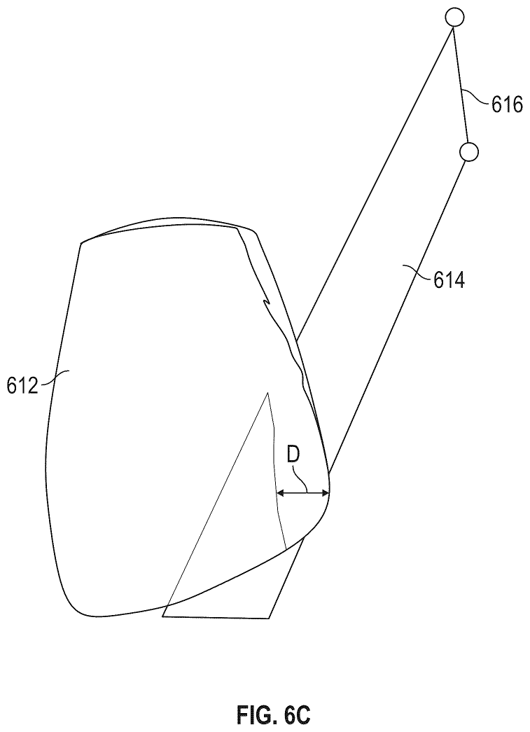

For example, FIG. 6A illustrates a 2D image 600a of a patient's teeth 602 and FIG. 6B illustrates a 3D model 600b of the patient's teeth 602, in accordance with embodiments of the present technology. The projection of the 2D image 600a in the 3D reference frame can be a 2D plane (“image plane 606”) within the 3D reference frame of the 3D model 600b. The 2D image 600a can be projected into the 3D reference frame by determining a camera position 604 such that a projection of the 3D model 600b onto a 2D plane matches or is similar to the 2D image 600a. In some embodiments, this process involves setting a camera position 604, projecting the 3D model 600b onto a 2D plane using the camera position 604 as the viewpoint for the projection, then comparing the 2D projection of the 3D model 600b to the 2D image 600a. If the 2D projection is sufficiently similar to the 2D image 600a, the camera position 604 is used as the projection viewpoint of the 2D image 600a, and the 2D plane is used as the image plane 606. Optionally, multiple 2D projections of the 3D model 600b can be generated from a plurality of different camera positions 604, and the camera position 604 and 2D plane that produce the 2D projection with the highest degree of similarity to the 2D image 600a can be selected as the projection viewpoint and image plane 606, respectively. Once the location of the image plane 606 in the 3D reference frame has been determined, each pixel location within the 2D image 600a can be mapped to a corresponding 3D coordinate location in the image plane 606, and thus, the 3D reference frame of the 3D model 600b.

Referring again to FIG. 5, at block 510, the method 500 can continue with generating a 3D projection of the at least one smile line, based on the correspondence between the 2D image and the 3D digital representation. As described herein, the smile line can be determined from the 2D image and thus can be initially defined with respect to the 2D reference frame of the 2D image (e.g., as a set of 2D coordinates). The 3D projection of the smile line can be a projection of the smile line into the 3D reference frame of the 3D digital representation. For instance, the 3D projection of the smile line 608 can be generated by using the correspondence determined in block 508 to map the 2D location of the smile line to the corresponding 3D location in the 3D reference frame. In some embodiments, the 3D projection of the smile line is a 2D plane (“smile plane”) in the 3D reference frame. As used herein, the term “smile plane” encompasses any surface in 3D space (e.g., a planar surface or a curved surface) that contains some or all of the 3D coordinates of the smile line, and that also intersects the projection viewpoint. In some embodiments, if the smile line is a curved line (e.g., a gingival line or an incisal edge line), the corresponding smile plane can be a curved surface that contains some or all of 3D coordinates of the curved line, and that also intersects the projection viewpoint. Alternatively, the smile plane corresponding to a curved smile line can be represented as a planar surface rather than a curved surface, for purposes of simplicity.

For example, as shown in FIG. 6A, one or more smile lines 608 can be generated from the 2D image 600a, such as a facial midline, ICW lines, gingival line, incisal edge line, horizontal line, and/or proportion lines. Each smile line 608 can include a plurality of 2D pixels defining the location and shape of the smile line 608. As shown in FIG. 6B, the locations of the 2D pixels of the smile lines 608 can be mapped to corresponding 3D coordinates in the image plane 606 in the 3D reference frame. Optionally, a 2D smile plane 610 can be determined for each smile line 608. The smile plane 610 can intersect the 3D locations of the smile line 608 on the image plane 606, and can also intersect the camera position 604 which correlates to the projection viewpoint for the image plane 606.

Referring again to FIG. 5, at block 512, the method 500 can include determining a target arrangement for the patient's teeth based on the 3D projection of the at least one smile line. In some embodiments, the process of block 512 involves determining a current spatial relationship between a tooth in the 3D digital representation and the 3D projection of the smile line, such as a current distance between the tooth and a smile plane corresponding to the smile line. The process can then involve comparing the current spatial relationship to a target spatial relationship between the tooth and the 3D projection of the smile line, such as a target distance between the tooth and the smile plane. For example, the target distance can be zero or approximately zero if the tooth is intended to touch, be tangential to, and/or intersect the smile line in the target arrangement. As another example, the target distance can be greater than zero if the tooth is intended to be spaced apart from the smile line in the target arrangement. If the current spatial relationship differs significantly from the target spatial relationship, the position of the tooth in the 3D digital representation can be adjusted to conform more closely to the target spatial relationship. For instance, if the current distance differs from the target distance by greater than a threshold value, the position of the tooth can be adjusted until the current distance falls within the threshold value.

For example, FIG. 6C illustrates a 3D model of a patient's tooth 612 together with a smile plane 614, in accordance with embodiments of the present technology. The smile plane 614 can be a 3D projection of a smile line 616, as described herein. The spatial relationship between the tooth 612 and the smile plane 614 can correlate to the spatial relationship between the tooth 612 and the smile line 616 in the 2D image, and thus can be used to determine how to adjust the patient's teeth to conform more closely to the target smile defined by the smile line 616.

For example, in some embodiments, the spatial relationship between the tooth 612 and the smile plane 614 is expressed in terms of a distance D between the tooth 612 and the smile plane 614. The distance D can be zero if the tooth 612 is aligned with (e.g., tangential to) the smile plane 614. The distance D can be positive if the tooth 612 intersects and protrudes past the smile plane 614, as shown in FIG. 6C. The distance D can be negative if the tooth 612 is spaced apart from the smile plane 614. The position of the tooth 612 in the target arrangement can be determined by comparing the distance D to a target distance between the tooth 612 and the smile plane 614. For instance, if the distance D between the tooth 612 and the smile plane 614 is greater than the target distance between the tooth 612 and the smile plane 614, the tooth 612 can be moved toward the left to reduce the extent to which the tooth 612 protrudes past the smile plane 614. Conversely, if the distance D is smaller than the target distance between the tooth 612 and the smile plane 614, the tooth 612 can be moved toward the right to decrease the gap between the tooth 612 and the smile plane 614.

Referring again to FIG. 5, in some embodiments, the process of block 512 is performed using a treatment planning algorithm that implements one or more rules for determining the positions of teeth in the target arrangement. The rules can include targets and/or constraints that are defined based on the smile lines, such as: a target position for a tooth relative to a smile line, a range of acceptable positions for a tooth relative to a smile line, a range of forbidden positions for a tooth relative to a smile line, a target distance between a tooth and a smile line, a range of acceptable distances between a tooth and a smile line, a range of forbidden distances between a tooth and a smile line, and so on. For example, a rule can indicate that a tooth should be aligned with, touch, and/or intersect a smile line. As another example, a rule can indicate that a tooth should not intersect a smile line. In a further example, a rule can indicate that a tooth should be within a certain distance of a smile line. In yet another example, a rule can indicate that tooth should be at least a certain distance away from a smile line. Any of the rules can be expressed in terms of a smile line, in terms of a 3D projection of the smile line (e.g., a smile plane), or both. Optionally, in embodiments where multiple rules are used, the rules can be weighted or otherwise prioritized differently, e.g., some rules may be mandatory while other rules may be optional.

Any of the rules herein can be applied to a single tooth, or can be applied to multiple teeth (e.g., two, three, four, five, or more teeth). In some embodiments, the rules may be applied only to one or more of the six anterior teeth of the patient's upper arch (e.g., central incisors, lateral incisors, and canines). In other embodiments, however, the rules can be applied to other teeth, such as other teeth of the patient's upper arch, and/or teeth of the patient's lower arch. Moreover, some teeth may be weighted or otherwise prioritized differently over other teeth, e.g., it may be mandatory for certain teeth to comply with a rule while it may be optional for other teeth to comply with the rule.

The rules herein can be expressed as one or more mathematical functions that are implemented by the treatment planning algorithm to determine the target arrangement. The functions can define a penalty parameter to be used in optimizing the positions of the teeth in the target arrangement. The penalty parameter can be indicative of the degree to which a tooth violates a particular rule. For instance, the penalty parameter can be larger if the current spatial relationship between a tooth and a smile plane differs significantly from the target spatial relationship specified by the rule, and can be smaller if the current spatial relationship between the tooth and the smile line matches or is close to the target spatial relationship. The treatment planning algorithm can determine the target positions of the teeth by reducing or minimizing the sum of the penalty parameter evaluated for all teeth.

For example, in some embodiments, the process of block 512 involves determining a distance between a tooth and a smile plane, which can be defined mathematically using the equation

$${D}_{s} ( p ) = ( \vec{p} - {\vec{p}}_{0} , \vec{n} )$$

where \vec{p} is a point on the tooth, \vec{p}₀ is a projection of the point onto the smile plane, and \vec{n} is the unit vector normal to the smile plane (e.g., the normal vector pointing away from the tooth).

An evaluator function that measures the amount of the tooth protruding outward past smile plane can be determined. The evaluator function can have the form

$$Penalty = {max}_{\vec{p} ∈ tooth} {D}_{s} ( \vec{p} )$$

and can be used as a target function or as a constraint.

In some embodiments, the shape of the tooth can be approximated using a plurality of 3D shapes, which can improve computational efficiency compared to approximation using a plurality of vertices. For example, the 3D shapes can be capsules that each have an elongate body and hemispherical ends. The tooth shape can thus be approximated by packing a portion of or the entirety of the tooth volume with the 3D shapes. The 3D shape can subsequently be used to calculate distances between the tooth and smile plane, e.g., by calculating the distance between the smile plane and the 3D shape(s) closest to the smile plane. Additional details of this approximation technique are provided in U.S. Pat. No. 11,096,763, the disclosure of which is incorporated by reference herein in its entirety.

For example, a distance between a smile plane and a tooth that is approximated with a plurality of capsules can be defined as

$${D}_{s} ( c ) = max ( {D}_{s} ( {\vec{o}}_{1} ) , {D}_{s} ( {\vec{o}}_{2} ) ) - r$$

where \vec{o}₁, \vec{o}₂ are the endpoints of a capsule segment and r is the radius of the capsule. The corresponding evaluator function can have the form

$$Penalty = {max}_{c ∈ approximation} {D}_{s} ( c )$$

and can be used as a target function or as a constraint.

In some embodiments, a rule for ensuring that a tooth does not protrude past a smile line more than a threshold value has the following form (Equation 1):

$$Penalty ( plane ) < T$$

where plane is the smile plane corresponding to the smile line, and T is the threshold value.

In some embodiments, a rule for ensuring that a space between a tooth and a smile line is less than a threshold value has the following form (Equation 2):

$$Penalty ( plane ) > - T$$

where plane is the smile plane corresponding to the smile line, and T is the threshold value.

In some embodiments, a rule for minimizing an amount of tooth protruding past a smile line has the following form (Equation 3):

$${max ( 0 , Penalty ( plane ) )}^{2}$$

where plane is the smile plane corresponding to the smile line.

In some embodiments, a rule for minimizing an amount of space between a tooth and a smile line has the following form (Equation 4):

$${min ( 0 , Penalty ( plane ) )}^{2}$$

where plane is the smile plane corresponding to the smile line.

For example, in the target arrangement, the right edge of the left central incisor can be aligned with the facial midline, while the left edge of the right central incisor can be aligned with the facial midline. The corresponding target function can be the sum of Equation 3 and Equation 4 for the left and right central incisors, using the smile plane corresponding to the facial midline. This target function can minimize the square distance between the central incisors and the facial midline, by locating the respective edges of these teeth as close to the facial midline as possible.

As another example, in the target arrangement, the incisal edges of the six upper anterior teeth can be aligned with the incisal edge line. The corresponding target function can be the sum of Equation 3 and Equation 4 for the six upper anterior teeth, using the smile plane corresponding to the incisal edge line, respectively. This target function can minimize the square distance between the six upper anterior teeth and the incisal edge line by locating the respective edges of these teeth as close to the incisal edge line. Alternatively or in combination, the gingival margin of the six upper anterior teeth can be aligned with the gingival line using this approach.

Once the target arrangement has been determined, the method 500 can proceed with generating one or more intermediate arrangements of the patient's teeth to reposition the teeth toward the target arrangement, and generating fabrication instructions for one or more dental appliances, as described elsewhere herein.

The method 500 can be varied in many ways. For example, some of the processes shown in FIG. 5 can be omitted and/or the method 500 can include additional processes not shown in FIG. 5. Moreover, the method 500 can be combined with any of the other methods described herein, such as the method 200 of FIG. 2.

FIGS. 7A-12 illustrate treatment planning techniques to reduce the amount of visible gingiva in a patient's smile, in accordance with embodiments of the present technology. A smile that exposes a significant amount of gingiva may be undesirable from an aesthetic standpoint, in some instances. For example, FIG. 7A illustrates a patient smile 700a with excess visible gingiva 702. The gingiva 702 between the patient's upper lip 704 and anterior teeth 706 is exposed in the smile 700a, which may not be aesthetically desirable. If aesthetic considerations are not taken into account during treatment planning, the amount of visible gingiva 702 may even be increased after orthodontic treatment, e.g., if the patient's upper anterior teeth 706 are extruded by the treatment.

FIG. 7B illustrates a patient smile 700b with reduced visible gingiva 708, in accordance with embodiments of the present technology. As shown in FIG. 7B, the gingiva 708 of the upper arch is now largely obscured by the patient's upper lip 704 when the patient smiles, which may be a more aesthetically pleasing result. The amount of exposed gingiva 708 can be decreased by reducing the extent to which the patient's anterior teeth 706 are extruded during treatment or by intruding the patient's anterior teeth 706. This result can be accomplished using an automated treatment planning algorithm that evaluates the amount of visible gingiva that is initially visible in the patient's smile and determines a target arrangement of the patient's teeth to maintain or reduce the amount of visible gingiva after treatment.

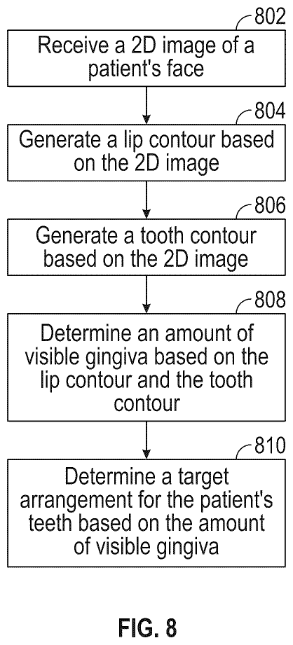

FIG. 8 is a flow diagram illustrating a method 800 for planning a treatment for a patient's teeth based on visible gingiva, in accordance with embodiments of the present technology. The method 800 can be performed by any suitable system or device, such as the by the data input component 102 and/or treatment planning component 104 of the system 100 of FIG. 1. In some embodiment, some or all of the processes of the method 800 are implemented as computer-readable instructions (e.g., program code) that are configured to be executed by one or more processors of a computing device.

The method 800 can begin at block 802 with receiving a 2D image of a patient's face. The 2D image (e.g., a photograph or an image frame of a video) can be received from any suitable imaging device, such as a camera (e.g., a DSLR camera or a camera of a mobile device). The 2D image can include the patient's mouth region, including the visible portions of the teeth and gingiva, as well as the patient's lips. The patient's mouth can be in any suitable position, such as a smiling position (e.g., a social smiling position). Optionally, the 2D image data can show other parts of the patient's anatomy, such as other facial features (e.g., eyes, eyebrows, nose, subnasion, cheeks, chin, jawline), head, neck, shoulders, and/or torso, or the entire body of the patient.

At block 804, the method 800 can include generating a lip contour based on the 2D image. The lip contour can be a spline, curve, or other geometric element representing the location and shape of at least one lip of the patient, such as the upper lip, the lower lip, or both lips. The lip contour can indicate the border of the lip that is proximate to the patient's teeth, e.g., the lower border of the upper lip and/or the upper border of the lower lip. The lip contour can be generated using any suitable technique, such as using a computer vision algorithm and/or machine learning algorithm to detect the locations of the lip(s) in the 2D image. Additional details of techniques for generating lip contours are provided in U.S. Pat. No. 11,007,036, the disclosure of which is incorporated by reference herein in its entirety.

At block 806, the method 800 can include generating a tooth contour based on the 2D image. The tooth contour can be a spline, curve, or other geometric element representing the location and shape of the gingival margins of one or more teeth, such as one or more anterior teeth of the upper arch (e.g., central incisors, lateral incisors, and/or canines), one or more anterior teeth of the lower arch, and/or other teeth that are visible in the 2D image. The tooth contour can be generated using any suitable technique, such as using a computer vision algorithm and/or machine learning algorithm to detect the locations of the teeth in the 2D image. Additional details of techniques for generating tooth contours are provided in U.S. Pat. No. 11,007,036, the disclosure of which is incorporated by reference herein in its entirety.

For example, FIG. 9 is a representative example of a patient image 900 annotated with a lip contour 902 and a tooth contour 904, in accordance with embodiments of the present technology. In the illustrated embodiment, the lip contour 902 corresponds to the lower edge of the upper lip of the patient, and the tooth contour 904 corresponds to the gingival margins of the six anterior teeth of the patient's upper arch. In other embodiments, however, a lip contour corresponding to the upper edge of the lower lip can be generated, alternatively or in addition to the lip contour 902. Moreover, the tooth contour 904 can be based on different teeth in the upper arch (e.g., the central incisors only), and/or a tooth contour can be generated for the teeth of the lower arch, alternatively or in addition to the tooth contour 904.

Referring again to FIG. 8, at block 808, the method 800 can continue with determining an amount of visible gingiva based on the lip contour and the tooth contour. For example, the amount of visible gingiva can be determined by measuring a distance between the lip contour and the tooth contour, which can correlate to the distance between the border of the patient's lip and the gingival margins of the patient's teeth. In some embodiments, the distance is a vertical distance between the lip contour and an apex of the tooth contour (corresponding to a gingival apex of a tooth). For example, as shown in FIG. 9, the amount of visible gingiva 906 between the patient's upper lip and upper teeth can be expressed in terms of distances D₁ and D₂, which are measured vertically between the lip contour 902 and the peaks of the tooth contour 904 corresponding to the gingival apices of the upper central incisors. Alternatively or in combination, distance measurements can be made at other locations along the tooth contour 904, such as at a peak corresponding to the gingival apex of another tooth (e.g., a lateral incisor or a canine).

Referring again to FIG. 8, the distance can be measured at a single location (e.g., from the gingival apex of a single tooth) or can be measured at multiple locations (e.g., from the gingival apices of multiple teeth). In embodiments where multiple distance measurements are made, the measurements can be averaged or otherwise combined with each other to generate a single value representing the overall amount of visible gingiva. Certain distance measurements may be weighted more heavily than other distance measurements, e.g., the measurements for the central incisors may be weighted more heavily than the measurements for the lateral incisors. Alternatively, the maximum distance measurement can be selected to the measurement representing the overall amount of visible gingiva.

Moreover, the process of block 808 can alternatively or additionally include other techniques for determining the amount of visible gingiva, besides distance-based measurements. For instance, the amount of visible gingiva can be determined by measuring an area between the lip contour and the tooth contour, such as an area corresponding to the area between the gingival margins of one or more teeth and the border of the patient's lip.

At block 810, the method 800 can continue with determining a target arrangement for the patient's teeth based on the amount of visible gingiva. In some embodiments, the process of block 810 involves comparing the amount of visible gingiva determined in block 808 to a threshold value. For instance, the threshold value can be a distance value representing the maximum distance of exposed gingiva that is considered to be acceptable, such as 5 mm, 4 mm, 3 mm, 2.5 mm, 2 mm, 1.5 mm, or 1 mm. As another example, the threshold value can be an area value representing the maximum area of the exposed gingiva that is considered to be acceptable.

If the amount of visible gingiva is less than or equal to the threshold value, the treatment planning algorithm can proceed with determining the positions of one or more teeth in the target arrangement such that the amount of visible gingiva remains the same, is decreased, or is increased but without exceeding the threshold value. For instance, the treatment planning algorithm can implement a rule indicating that the maximum extrusion distance of the anterior teeth is no more than the difference between the maximum acceptable distance of the visible gingiva and the current measured distance of the visible gingiva (e.g., if the maximum acceptable distance is 3 mm and the current measured distance is 2 mm, the maximum extrusion distance should be no more than 1 mm).

If the amount of visible gingiva exceeds the threshold, the treatment planning algorithm can proceed with determining the positions of one or more teeth in the target arrangement to decrease the amount of visible gingiva. For instance, the treatment planning algorithm can intrude one or more teeth to reduce the distance of exposed gingiva after treatment. The intrusion distance can be determined based on the difference between the maximum acceptable distance of the visible gingiva and the current measured distance of the visible gingiva (e.g., if the maximum acceptable distance is 2 mm and the current measured distance is 3 mm, the intrusion distance should be at least 1 mm). Optionally, the intrusion distance can be constrained to be no more than a maximum value corresponding to clinically acceptable movement limits (e.g., no more than 2 mm).

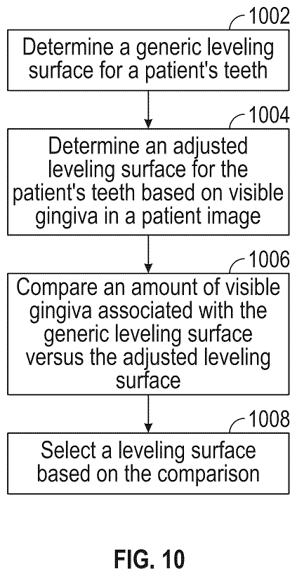

In some embodiments, the treatment planning algorithm determines the target arrangement of the teeth based in part on a leveling surface that defines the vertical positions of one or more teeth. The parameters of the leveling surface can be determined based on the amount of visible gingiva. For instance, the parameters of the leveling surface can be configured to increase, decrease, or maintain the amount of visible gingiva. Accordingly, the process of block 810 can involve determining a leveling surface that produces the desired amount of visible gingiva when the patient's teeth are in the target arrangement, as described further below in connection with FIGS. 10-12.

Once the target arrangement has been determined, the method 800 can proceed with generating one or more intermediate arrangements of the patient's teeth to reposition the teeth toward the target arrangement, and generating fabrication instructions for one or more dental appliances, as described elsewhere herein.

The method 800 can be varied in many ways. For example, some of the processes shown in FIG. 8 can be omitted and/or the method 800 can include additional processes not shown in FIG. 8. Moreover, the method 800 can be combined with any of the other methods described herein, such as the method 200 of FIG. 2 and/or the method 500 of FIG. 5.

FIG. 10 is a flow diagram illustrating a method 1000 for determining a target arrangement of a patient's teeth to reduce visible gingiva, in accordance with embodiments of the present technology. The method 1000 can be performed by any suitable system or device, such as the by the data input component 102 and/or treatment planning component 104 of the system 100 of FIG. 1. In some embodiment, some or all of the processes of the method 1000 are implemented as computer-readable instructions (e.g., program code) that are configured to be executed by one or more processors of a computing device.

The method 1000 can begin at block 1002 with determining a generic leveling surface of a patient's teeth. The generic leveling surface can be a plane or other geometric element that indicates target vertical positions for one or more teeth, e.g., the cusps of the teeth can be constrained to lie on the generic leveling surface. For instance, the generic leveling surface can indicate the vertical positions of the incisors, canines, and/or premolars of the patient's upper arch to be achieved through orthodontic treatment. In some embodiments, the vertical position of the generic leveling surface is determined based on a current vertical position of one or more teeth (e.g., the premolars), and is used to specify a target vertical position for one or more other teeth (e.g., the central incisors, lateral incisors, and/or canines). The shape of the generic leveling surface can set how the teeth are positioned vertically relatively to each other, e.g., the teeth can have the same vertical positions if the generic leveling surface is a horizontal plane, or can have different vertical positions if the generic leveling surface is an angled plane or a curved surface.

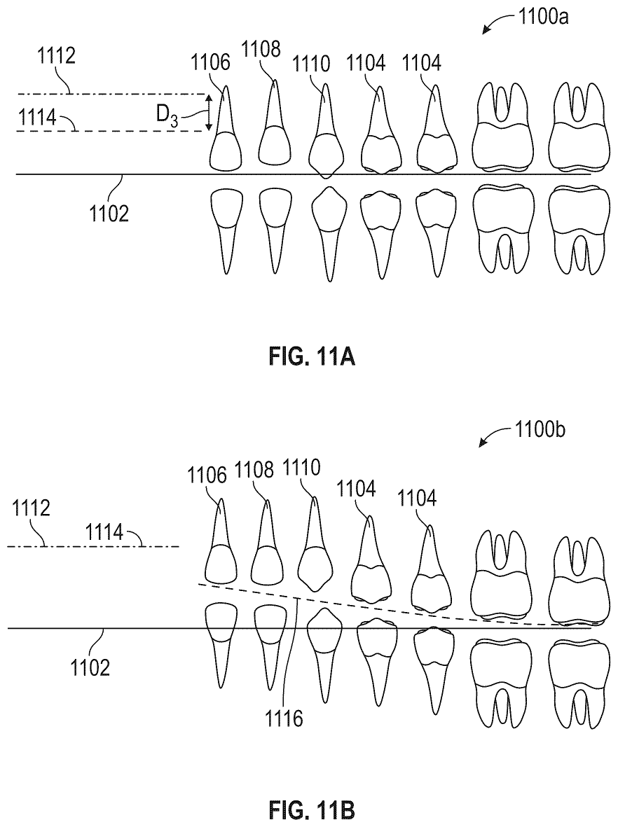

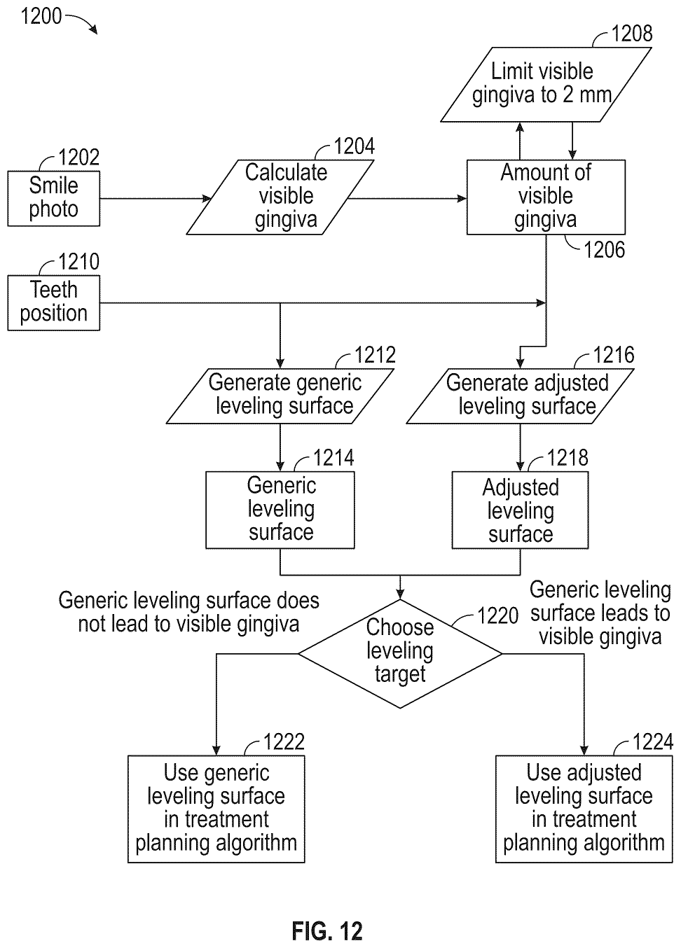

In some embodiments, the generic leveling surface is a “default” leveling surface that is determined independently of the amount of visible gingiva in the patient's smile. Accordingly, repositioning the patient's teeth according to the generic leveling surface may result in excess visible gingiva. For example, FIG. 11A is a schematic illustration of a patient's teeth in a target arrangement 1100a corresponding to a generic leveling surface 1102, in accordance with embodiments of the present technology. The vertical position (height) of the generic leveling surface 1102 can be set based on the current vertical positions of one or more premolars of the patient's upper arch. For instance, the generic leveling surface 1102 can be aligned with (e.g., intersect or be tangential to) the cusps of the premolars 1104. The generic leveling surface 1102 can be used to set the vertical positions of the central incisors 1106 in the target arrangement 1100a, e.g., the central incisors 1106 can be aligned with (e.g., intersect or be tangential to) the generic leveling surface 1102. Alternatively or in combination, the generic leveling surface 1102 can be used to set the vertical positions of other teeth, such as the lateral incisors 1108 and/or canines 1110.

When the patient's teeth are in the target arrangement 1100a, the amount of visible gingiva can correspond to a distance D₃ between the vertical position of the lower edge of the patient's upper lip (“lip position 1112”) and the vertical position of the gingival margin of one or more anterior teeth such the central incisors 1106 (“gingival margin 1114”). Accordingly, if the anterior teeth are extruded in the target arrangement 1100a in order to be aligned with the generic leveling surface 1102, this may increase the distance D₃ and, thus, the amount of exposed visible gingiva.

Referring again to FIG. 10, at block 1004, the method 1000 can include determining an adjusted leveling surface for the patient's teeth based on visible gingiva in a patient image. The adjusted leveling surface can be a plane or other geometric element that indicates target vertical positions for one or more teeth, e.g., the cusps of the teeth can be constrained to lie on the generic leveling surface. For instance, the adjusted leveling surface can indicate the vertical positions of the incisors, canines, and/or premolars of the patient's upper arch to be achieved through orthodontic treatment. The shape of the adjusted leveling surface can set how the teeth are positioned vertically relatively to each other, e.g., the teeth can have the same vertical positions if the generic leveling surface is a horizontal plane, or can have different vertical positions if the generic leveling surface is an angled plane or a curved surface.

The adjusted leveling surface can be determined based on the amount of visible gingiva in the patient image, which can be measured using the techniques previously described herein (e.g., in connection with blocks 802-808 of FIG. 8). In some embodiments, the adjusted leveling surface specifies target vertical positions for one or more teeth (e.g., central incisors, lateral incisors, and/or canines) that would reduce the amount of visible gingiva in the patient's smile, e.g., to a vertical distance less than or equal to a threshold value. Optionally, the adjusted leveling surface can be constrained to avoid tooth movements that exceed clinically acceptable movement limits (e.g., no more than 2 mm of intrusion).

For example, FIG. 11B is a schematic illustration of a patient's teeth in a target arrangement 1100a corresponding to an adjusted leveling surface 1116, in accordance with embodiments of the present technology. In the illustrated embodiment, the adjusted leveling surface 1116 is a curved surface that is aligned with the cusps of the premolars 1104. The adjusted leveling surface 1116 can set the vertical positions of the central incisors 1106 in the target arrangement 1100b so that the distance between the lip position 1112 and the gingival margin 1114 of the central incisors 1106 is reduced or minimized (e.g., to zero as shown in FIG. 11B), thus decreasing the amount of visible gingiva. Optionally, the adjusted leveling surface 1116 can also set the vertical positions of the lateral incisors 1108 and/or canines 1110, e.g., by a defining a curve between the cusps of the central incisors 1106 and the cusps of the premolars 1104. As shown in FIG. 11B, the adjusted leveling surface 1116 can result in intrusion of the central incisors 1106, lateral incisors 1108, and/or canines 1110, compared to the generic leveling surface 1102.

Referring again to FIG. 10, at block 1006, the method 1000 can further include comparing an amount of visible gingiva associated with the generic leveling surface versus the adjusted leveling surface. In some embodiments, the process of block 1006 includes determining a first amount of visible gingiva that would result from repositioning the patient's teeth according to the generic leveling surface, determining a second amount of visible gingiva that would result from repositioning the patient's teeth according to the adjusted leveling surface, and comparing the first amount of visible gingiva to the second amount of visible gingiva. The amount of visible gingiva can be determined using distance measurements, area measurements, and/or other suitable techniques, as previously described in connection with FIG. 8. Optionally, the amount of visible gingiva after repositioning can be calculated be based on the amount of visible gingiva that is initially present and the change in the positions of one or more teeth when repositioned (e.g., if 1 mm of gingiva is initially visible and the tooth is extruded by 1 mm, the resulting amount of visible gingiva would be 2 mm.)

At block 1008, the method 1000 can continue with selecting a leveling surface (the generic leveling surface or the adjusted leveling surface) based on the comparison. For example, the leveling surface that results in the least amount of visible gingiva can be selected. As another example, the generic leveling surface can be selected if the generic leveling surface would not increase the amount of visible gingiva relative to the initial arrangement of the patient's teeth, whereas the adjusted leveling surface can be selected if the generic leveling surface would increase the amount of visible gingiva. In yet another example, the generic leveling surface can be selected if the generic leveling surface results in an amount of visible gingiva that does not exceed a threshold value, whereas the adjusted leveling surface can be selected if the generic leveling surface would result in an amount of visible gingiva that exceeds the threshold value.

Subsequently, the selected leveling surface can be used to determine a target arrangement of the patient's teeth, e.g., by inputting the selected leveling surface into a treatment planning algorithm. The target arrangement can then be used to determine intermediate arrangements and/or fabrication instructions for dental appliances, as described elsewhere herein.

The method 1000 can be varied in many ways. For example, some of the processes shown in FIG. 10 can be omitted and/or the method 1000 can include additional processes not shown in FIG. 10. Moreover, the method 1000 can be combined with any of the other methods described herein, such as the method 200 of FIG. 2, the method 500 of FIG. 5, and/or the method 800 of FIG. 8. For instance, the method 1000 can be performed as part of the process of block 810 of the method 800 of FIG. 8.

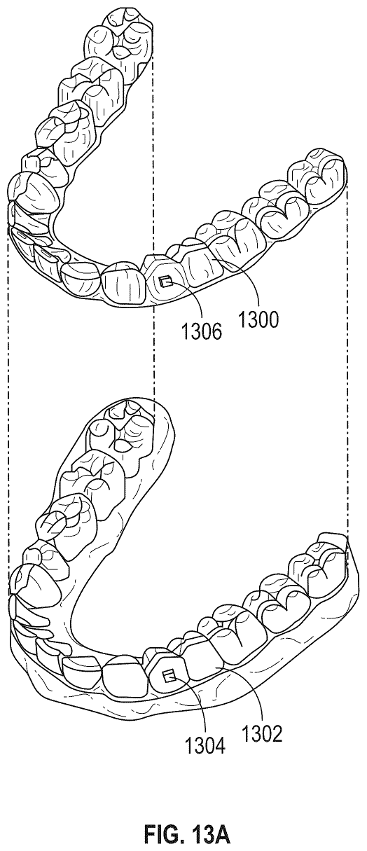

FIG. 12 is a flow diagram illustrating a workflow 1200 for generating a treatment plan to reduce visible gingiva, in accordance with embodiments of the present technology. The workflow 1200 can be implemented by any suitable system or device, such as the by the data input component 102 and/or treatment planning component 104 of the system 100 of FIG. 1. In some embodiment, some or all of the processes of the workflow 1200 are implemented as computer-readable instructions (e.g., program code) that are configured to be executed by one or more processors of a computing device.

The workflow 1200 can include receiving a photograph of a patient smiling (“smile photo,” block 1202) and calculating the amount of visible gingiva in the smile photo (blocks 1204, 1206). Optionally, if the amount of visible gingiva in the smile photo exceeds 2 mm, the amount used in subsequent processes can be set to 2 mm in order to remain within clinically acceptable tooth movement limits (block 1208).

The workflow 1200 can also include receiving data indicating the positions of one or more of the patient's teeth in an initial arrangement (“teeth position,” block 1210). A generic leveling surface for the patient's teeth can be generated based on the teeth position (blocks 1212, 1214). An adjusted leveling surface can be generated based on the teeth position and the amount of visible gingiva (blocks 1216, 1218).

The workflow 1200 can further include choosing a leveling target for the treatment planning algorithm (block 1220). In some embodiments, if the generic leveling surface does not result in visible gingiva in the patient's smile or does not increase the amount of visible gingiva in the patient's smile, the generic leveling surface is used in the treatment planning algorithm (block 1222). Conversely, if the generic leveling surface does result in visible gingiva and/or increases the amount of visible gingiva, the adjusted leveling surface is used in the treatment planning algorithm block 1224.

The workflow 1200 can be varied in many ways. For example, some of the inputs, outputs, and/or processes shown in FIG. 12 can be omitted, and/or the workflow 1200 can include additional inputs, outputs, and/or processes not shown in FIG. 12. Moreover, the workflow 1200 can be incorporated into any of the other methods described herein, such as the method 200 of FIG. 2, the method 500 of FIG. 5, the method 800 of FIG. 8, and/or the method 1000 of FIG. 10.

### II. Dental Appliances and Associated Methods

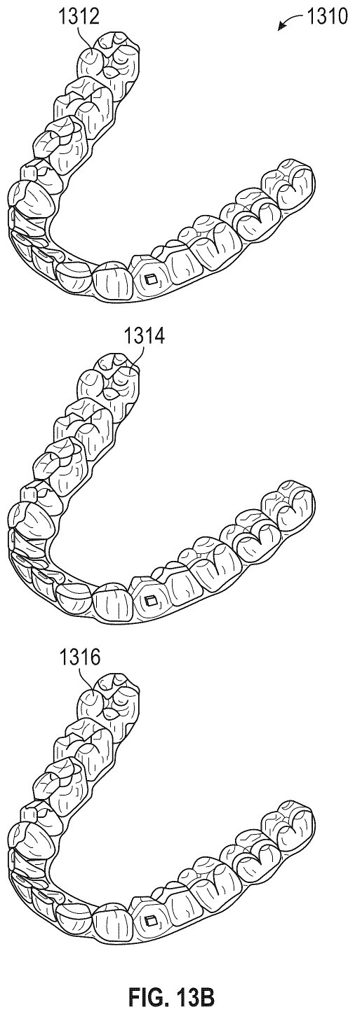

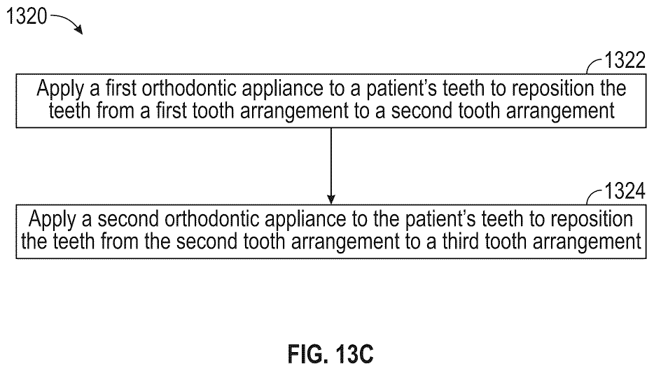

FIG. 13A illustrates a representative example of a tooth repositioning appliance 1300 configured in accordance with embodiments of the present technology. The appliance 1300 can be manufactured using any of the systems, methods, and devices described herein. The appliance 1300 (also referred to herein as an “aligner”) can be worn by a patient in order to achieve an incremental repositioning of individual teeth 1302 in the jaw. The appliance 1300 can include a shell (e.g., a continuous polymeric shell or a segmented shell) having teeth-receiving cavities that receive and resiliently reposition the teeth. The appliance 1300 or portion(s) thereof may be indirectly fabricated using a physical model of teeth. For example, an appliance (e.g., polymeric appliance) can be formed using a physical model of teeth and a sheet of suitable layers of polymeric material. In some embodiments, a physical appliance is directly fabricated, e.g., using additive manufacturing techniques, from a digital model of an appliance.

The appliance 1300 can fit over all teeth present in an upper or lower jaw, or less than all of the teeth. The appliance 1300 can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance 1300 can be a generic appliance configured to receive the teeth, but not necessarily shaped to match the topography of the patient's teeth. In some cases, only certain teeth received by the appliance 1300 are repositioned by the appliance 1300 while other teeth can provide a base or anchor region for holding the appliance 1300 in place as it applies force against the tooth or teeth targeted for repositioning. In some cases, some, most, or even all of the teeth can be repositioned at some point during treatment. Teeth that are moved can also serve as a base or anchor for holding the appliance as it is worn by the patient. In preferred embodiments, no wires or other means are provided for holding the appliance 1300 in place over the teeth. In some cases, however, it may be desirable or necessary to provide individual attachments 1304 or other anchoring elements on teeth 1302 with corresponding receptacles 1306 or apertures in the appliance 1300 so that the appliance 1300 can apply a selected force on the tooth. Representative examples of appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the url “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 13B illustrates a tooth repositioning system 1310 including a plurality of appliances 1312, 1314, 1316, in accordance with embodiments of the present technology. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 1310 can include a first appliance 1312 corresponding to an initial tooth arrangement, one or more intermediate appliances 1314 corresponding to one or more intermediate arrangements, and a final appliance 1316 corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

FIG. 13C illustrates a method 1320 of orthodontic treatment using a plurality of appliances, in accordance with embodiments of the present technology. The method 1320 can be practiced using any of the appliances or appliance sets described herein. In block 1322, a first orthodontic appliance is applied to a patient's teeth in order to reposition the teeth from a first tooth arrangement to a second tooth arrangement. In block 1324, a second orthodontic appliance is applied to the patient's teeth in order to reposition the teeth from the second tooth arrangement to a third tooth arrangement. The method 1320 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

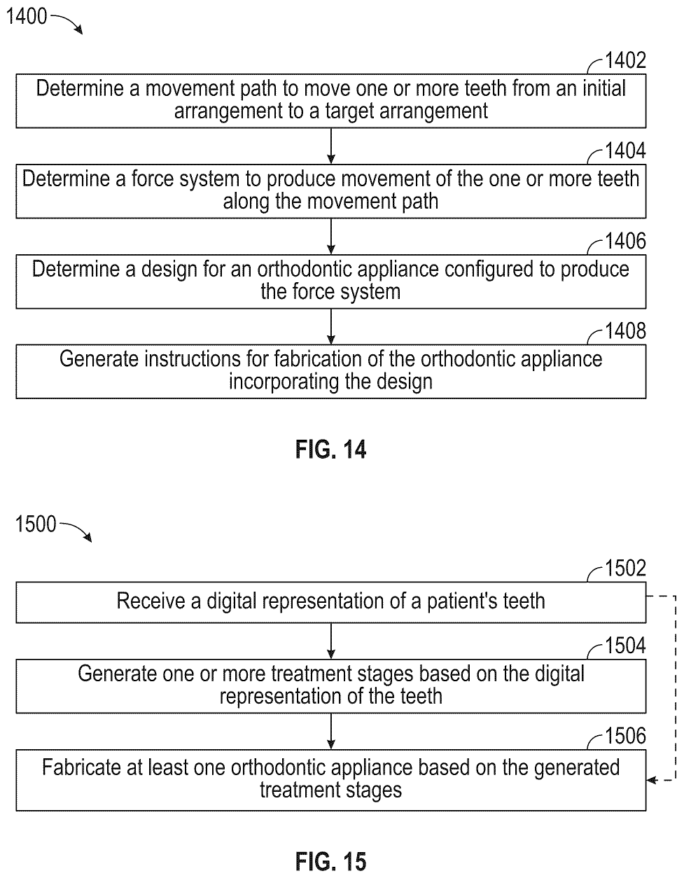

FIG. 14 illustrates a method 1400 for designing an orthodontic appliance, in accordance with embodiments of the present technology. The method 1400 can be applied to any embodiment of the orthodontic appliances described herein. Some or all of the steps of the method 1400 can be performed by any suitable data processing system or device, e.g., one or more processors configured with suitable instructions.

In block 1402, a movement path to move one or more teeth from an initial arrangement to a target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. From the obtained data, a digital data set can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, including measured or extrapolated hidden surfaces and root structures, as well as surrounding bone and soft tissue.

The target arrangement of the teeth (e.g., a desired and intended end result of orthodontic treatment) can be received from a clinician in the form of a prescription, can be calculated from basic orthodontic principles, and/or can be extrapolated computationally from a clinical prescription. With a specification of the desired final positions of the teeth and a digital representation of the teeth themselves, the final position and surface geometry of each tooth can be specified to form a complete model of the tooth arrangement at the desired end of treatment.

Having both an initial position and a target position for each tooth, a movement path can be defined for the motion of each tooth. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In block 1404, a force system to produce movement of the one or more teeth along the movement path is determined. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

Determination of the force system can be performed in a variety of ways. For example, in some embodiments, the force system is determined on a patient-by-patient basis, e.g., using patient-specific data. Alternatively or in combination, the force system can be determined based on a generalized model of tooth movement (e.g., based on experimentation, modeling, clinical data, etc.), such that patient-specific data is not necessarily used. In some embodiments, determination of a force system involves calculating specific force values to be applied to one or more teeth to produce a particular movement. Alternatively, determination of a force system can be performed at a high level without calculating specific force values for the teeth. For instance, block 1404 can involve determining a particular type of force to be applied (e.g., extrusive force, intrusive force, translational force, rotational force, tipping force, torquing force, etc.) without calculating the specific magnitude and/or direction of the force.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be measured, or input by a treating professional. In other embodiments, the treating professional can select an appropriate treatment based on physiological characteristics of the patient. For example, the properties of the palate may also be estimated based on factors such as the patient's age—for example, young juvenile patients can require lower forces to expand the suture than older patients, as the suture has not yet fully formed.

In block 1406, a design for an orthodontic appliance configured to produce the force system is determined. The design can include the appliance geometry, material composition and/or material properties, and can be determined in various ways, such as using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA (Abaqus) software products from Dassault Systèmes of Waltham, MA.

Optionally, one or more designs can be selected for testing or force modeling. As noted above, a desired tooth movement, as well as a force system required or desired for eliciting the desired tooth movement, can be identified. Using the simulation environment, a candidate design can be analyzed or modeled for determination of an actual force system resulting from use of the candidate appliance. One or more modifications can optionally be made to a candidate appliance, and force modeling can be further analyzed as described, e.g., in order to iteratively determine an appliance design that produces the desired force system.

In block 1408, instructions for fabrication of the orthodontic appliance incorporating the design are generated. The instructions can be configured to control a fabrication system or device in order to produce the orthodontic appliance with the specified design. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by thermoforming.

Although the above steps show a method 1400 of designing an orthodontic appliance in accordance with some embodiments, a person of ordinary skill in the art will recognize some variations based on the teaching described herein. Some of the steps may comprise sub-steps. Some of the steps may be repeated as often as desired. One or more steps of the method 1400 may be performed with any suitable fabrication system or device, such as the embodiments described herein. Some of the steps may be optional, e.g., the process of block 1404 can be omitted, such that the orthodontic appliance is designed based on the desired tooth movements and/or determined tooth movement path, rather than based on a force system. Moreover, the order of the steps can be varied as desired.

FIG. 15 illustrates a method 1500 for digitally planning an orthodontic treatment and/or design or fabrication of an appliance, in accordance with embodiments. The method 1500 can be applied to any of the treatment procedures described herein and can be performed by any suitable data processing system.

In block 1502 a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 1504, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 1506, at least one orthodontic appliance is fabricated based on the generated treatment stages. For example, a set of appliances can be fabricated, each shaped according to a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired.

In some instances, staging of various arrangements or treatment stages may not be necessary for design and/or fabrication of an appliance. As illustrated by the dashed line in FIG. 15, design and/or fabrication of an orthodontic appliance, and perhaps a particular orthodontic treatment, may include use of a representation of the patient's teeth (e.g., including receiving a digital representation of the patient's teeth (block 1502)), followed by design and/or fabrication of an orthodontic appliance based on a representation of the patient's teeth in the arrangement represented by the received representation.

As noted herein, the techniques described herein can be used for the direct fabrication of dental appliances, such as aligners and/or a series of aligners with tooth-receiving cavities configured to move a person's teeth from an initial arrangement toward a target arrangement in accordance with a treatment plan. Aligners can include mandibular repositioning elements, such as those described in U.S. Pat. No. 10,912,629, entitled “Dental Appliances with Repositioning Jaw Elements,” filed Nov. 30, 2015; U.S. Pat. No. 10,537,406, entitled “Dental Appliances with Repositioning Jaw Elements,” filed Sep. 19, 2014; and U.S. Pat. No. 9,844,424, entitled “Dental Appliances with Repositioning Jaw Elements,” filed Feb. 21, 2014; all of which are incorporated by reference herein in their entirety.

The techniques used herein can also be used to manufacture attachment placement devices, e.g., appliances used to position prefabricated attachments on a person's teeth in accordance with one or more aspects of a treatment plan. Examples of attachment placement devices (also known as “attachment placement templates” or “attachment fabrication templates”) can be found at least in: U.S. application Ser. No. 17/249,218, entitled, “Flexible 3D Printed Orthodontic Device,” filed Feb. 24, 2021; U.S. application Ser. No. 16/366,686, entitled “Dental Attachment Placement Structure,” filed Mar. 27, 2019; U.S. application Ser. No. 15/674,662, entitled “Devices and Systems for Creation of Attachments,” filed Aug. 11, 2017; U.S. Pat. No. 11,103,330, entitled “Dental Attachment Placement Structure,” filed Jun. 14, 2017; U.S. application Ser. No. 14/963,527, entitled “Dental Attachment Placement Structure,” filed Dec. 9, 2015; U.S. application Ser. No. 14/939,246, entitled “Dental Attachment Placement Structure,” filed Nov. 12, 2015; U.S. application Ser. No. 14/939,252, entitled “Dental Attachment Formation Structures,” filed Nov. 12, 2015; and U.S. Pat. No. 9,700,385, entitled “Attachment Structure,” filed Aug. 22, 2014; all of which are incorporated by reference herein in their entirety.

The techniques described herein can be used to make incremental palatal expanders and/or a series of incremental palatal expanders used to expand a person's palate from an initial position toward a target position in accordance with one or more aspects of a treatment plan. Examples of incremental palatal expanders can be found at least in: U.S. application Ser. No. 16/380,801, entitled “Releasable Palatal Expanders,” filed Apr. 10, 2019; U.S. application Ser. No. 16/022,552, entitled “Devices, Systems, and Methods for Dental Arch Expansion,” filed Jun. 28, 2018; U.S. Pat. No. 11,045,283, entitled, “Palatal Expander with Skeletal Anchorage Devices,” filed Jun. 8, 2018; U.S. application Ser. No. 15/831,159, entitled “Palatal Expanders and Methods of Expanding a Palate,” filed Dec. 4, 2017; U.S. Pat. No. 10,993,783, entitled “Methods and Apparatuses for Customizing a Rapid Palatal Expander,” filed Dec. 4, 2017; and U.S. Pat. No. 7,192,273, entitled “System and Method for Palatal Expansion,” filed Aug. 7, 2003; all of which are incorporated by reference herein in their entirety.

### Examples

The following examples are included to further describe some aspects of the present technology, and should not be used to limit the scope of the technology.

Example 1. A method comprising:
receiving a 2D image of a patient's face;
generating at least one smile line based on the 2D image;
receiving a 3D digital representation of the patient's arch;
determining a correspondence between the 2D image and the 3D digital representation; generating a 3D projection of the at least one smile line based on the correspondence; and determining a target arrangement for the patient's teeth based on the 3D projection of the at least one smile line.

Example 2. The method of Example 1, wherein the 3D projection of the at least one smile line comprises at least one smile plane.

Example 3. The method of Example 2, wherein the at least one smile plane contains the at least one smile line and intersects a projection viewpoint.

Example 4. The method of Example 3, wherein the projection viewpoint corresponds to a camera position that, when used to project the 3D digital representation onto a 2D plane, produces a 2D projection of the 3D digital representation that is similar to the 2D image.

Example 5. The method of any one of Examples 1 to 4, wherein the 2D image and the at least one smile line are in a 2D reference frame, and the 3D digital representation and the 3D projection of the at least one smile line are in a 3D reference frame.

Example 6. The method of any one of Examples 1 to 5, further comprising determining a current spatial relationship between at least one tooth of the 3D digital representation and the 3D projection of the at least one smile line.

Example 7. The method of Example 6, further comprising comparing the current spatial relationship to a target spatial relationship between the at least one tooth and the 3D projection.

Example 8. The method of Example 7, wherein:
the current spatial relationship comprises a current distance between the at least one tooth and the 3D projection, and
the target spatial relationship comprises a target distance between the at least one tooth and the 3D projection.

Example 9. The method of Example 7 or 8, further comprising adjusting a position of the at least one tooth in the target arrangement if the current spatial relationship differs from the target spatial relationship.

Example 10. The method of any one of Examples 6 to 9, wherein the at least one tooth comprises one or more of a central incisor, a lateral incisor, or a canine.

Example 11. The method of any one of Examples 1 to 10, wherein the target arrangement is determined using an automated treatment planning algorithm.

Example 12. The method of Example 11, wherein the automated treatment planning algorithm implements one or more rules for determining positions of the patient's teeth in the target arrangement.

Example 13. The method of Example 12, wherein the one or more rules are based on the at least one smile line.

Example 14. The method of Example 12 or 13, wherein the one or more rules define a penalty parameter based on a spatial relationship between at least one tooth and the 3D projection of the at least one smile line.

Example 15. The method of any one of Examples 1 to 14, wherein the 2D image is in a 2D reference frame, the 3D digital representation is in a 3D reference frame, and the correspondence comprises a mapping between the 2D reference frame and the 3D reference frame.

Example 16. The method of any one of Examples 1 to 15, wherein determining the correspondence between the 2D image and the 3D digital representation comprises:
projecting the 3D digital representation onto a 2D plane, and
comparing the projection of the 3D digital representation with the 2D image.

Example 17. The method of any one of Examples 1 to 16, wherein the 2D image shows the patient's mouth in a smiling position.

Example 18. The method of any one of Examples 1 to 17, wherein the 2D image is a photograph.

Example 19. The method of any one of Examples 1 to 18, wherein the 2D image is received from a mobile device.

Example 20. The method of any one of Examples 1 to 19, wherein the at least one smile line comprises one or more of the following: a facial midline, an intercanine width line, a gingival line, an incisal edge line, a horizontal line, or a proportion line.

Example 21. The method of any one of Examples 1 to 20, wherein the at least one smile line defines a target smile for the patient.

Example 22. The method of any one of Examples 1 to 21, further comprising identifying one or more facial landmarks in the 2D image, wherein the at least one smile line is generated based on the one or more facial landmarks.

Example 23. The method of any one of Examples 1 to 22, wherein the 3D digital representation comprises a 3D digital model of one or more teeth of the patient's arch in an initial arrangement.

Example 24. The method of any one of Examples 1 to 23, wherein the patient's arch is an upper arch.

Example 25. The method of any one of Examples 1 to 24, further comprising generating one or more intermediate arrangements to reposition the patient's teeth toward the target arrangement.

Example 26. The method of any one of Examples 1 to 25, further comprising generating instructions for fabricating one or more dental appliances configured to reposition the patient's teeth toward the target arrangement.

Example 27. A system comprising:
a processor; and
a memory operably coupled to the processor and storing instructions that, when executed by the processor, cause the system to perform operations comprising:
   receiving a 2D image of a patient's face;
   generating at least one smile line based on the 2D image;
   receiving a 3D digital representation of the patient's arch;
   determining a correspondence between the 2D image and the 3D digital representation;
   generating a 3D projection of the at least one smile line based on the correspondence; and
   determining a target arrangement for the patient's teeth based on the 3D projection of the at least one smile line.

Example 28. The system of Example 27, wherein the 3D projection of the at least one smile line comprises at least one smile plane.

Example 29. The system of Example 28, wherein the at least one smile plane contains the at least one smile line and intersects a projection viewpoint.

Example 30. The system of Example 29, wherein the projection viewpoint corresponds to a camera position that, when used to project the 3D digital representation onto a 2D plane, produces a 2D projection of the 3D digital representation that is similar to the 2D image.

Example 31. The system of any one of Examples 27 to 30, wherein the 2D image and the at least one smile line are in a 2D reference frame, and the 3D digital representation and the 3D projection of the at least one smile line are in a 3D reference frame.

Example 32. The system of any one of Examples 27 to 31, wherein the operations further comprise determining a current spatial relationship between at least one tooth of the 3D digital representation and the 3D projection of the at least one smile line.

Example 33. The system of Example 32, wherein the operations further comprise comparing the current spatial relationship to a target spatial relationship between the at least one tooth and the 3D projection.

Example 34. The system of Example 33, wherein:
the current spatial relationship comprises a current distance between the at least one tooth and the 3D projection, and
the target spatial relationship comprises a target distance between the at least one tooth and the 3D projection.

Example 35. The system of Example 33 or 34, wherein the operations further comprise adjusting a position of the at least one tooth in the target arrangement if the current spatial relationship differs from the target spatial relationship.

Example 36. The system of any one of Examples 32 to 35, wherein the at least one tooth comprises one or more of a central incisor, a lateral incisor, or a canine.

Example 37. The system of any one of Examples 27 to 36, wherein the target arrangement is determined using an automated treatment planning algorithm.

Example 38. The system of Example 37, wherein the automated treatment planning algorithm implements one or more rules for determining positions of the patient's teeth in the target arrangement.

Example 39. The system of Example 38, wherein the one or more rules are based on the at least one smile line.

Example 40. The system of Example 38 or 39, wherein the one or more rules define a penalty parameter based on a spatial relationship between at least one tooth and the 3D projection of the at least one smile line.

Example 41. The system of any one of Examples 27 to 40, wherein the 2D image is in a 2D reference frame, the 3D digital representation is in a 3D reference frame, and the correspondence comprises a mapping between the 2D reference frame and the 3D reference frame.

Example 42. The system of any one of Examples 27 to 41, wherein determining the correspondence between the 2D image and the 3D digital representation comprises:
projecting the 3D digital representation onto a 2D plane, and
comparing the projection of the 3D digital representation with the 2D image.

Example 43. The system of any one of Examples 27 to 42, wherein the 2D image shows the patient's mouth in a smiling position.

Example 44. The system of any one of Examples 27 to 43, wherein the 2D image is a photograph.

Example 45. The system of any one of Examples 27 to 44, wherein the 2D image is received from a mobile device.

Example 46. The system of any one of Examples 27 to 45, wherein the at least one smile line comprises one or more of the following: a facial midline, an intercanine width line, a gingival line, an incisal edge line, a horizontal line, or a proportion line.

Example 47. The system of any one of Examples 27 to 46, wherein the at least one smile line defines a target smile for the patient.

Example 48. The system of any one of Examples 27 to 47, wherein the operations further comprise identifying one or more facial landmarks in the 2D image, wherein the at least one smile line is generated based on the one or more facial landmarks.

Example 49. The system of any one of Examples 27 to 48, wherein the 3D digital representation comprises a 3D digital model of one or more teeth of the patient's arch in an initial arrangement.

Example 50. The system of any one of Examples 27 to 49, wherein the patient's arch is an upper arch.

Example 51. The system of any one of Examples 27 to 50, wherein the operations further comprise generating one or more intermediate arrangements to reposition the patient's teeth toward the target arrangement.

Example 52. The system of any one of Examples 27 to 51, wherein the operations further comprise generating instructions for fabricating one or more dental appliances configured to reposition the patient's teeth toward the target arrangement.

Example 53. A non-transitory computer-readable storage medium comprising instructions that, when executed by one or more processors of a computing system, cause the computing system to perform operations comprising:
receiving a 2D image of a patient's face;
generating at least one smile line based on the 2D image;
receiving a 3D digital representation of the patient's arch;
determining a correspondence between the 2D image and the 3D digital representation;
generating a 3D projection of the at least one smile line based on the correspondence; and
determining a target arrangement for the patient's teeth based on the 3D projection of the at least one smile line.

Example 54. A method comprising:
receiving a 2D image of a patient's face;
determining an amount of visible gingiva in the 2D image; and
determining a target arrangement for the patient's teeth based on the amount of visible gingiva.

Example 55. The method of Example 54, further comprising generating a lip contour and a tooth contour based on the 2D image, wherein the amount of visible gingiva is determined based on the lip contour and the tooth contour.

Example 56. The method of Example 55, wherein the lip contour corresponds to a border of a lip of the patient.

Example 57. The method of Example 56, wherein the lip contour corresponds to a lower border of an upper lip of the patient.

Example 58. The method of any one of Examples 55 to 57, wherein the tooth contour corresponds to a gingival margin of at least one tooth of the patient.

Example 59. The method of Example 58, wherein the at least one tooth comprises an anterior tooth of the patient's upper arch.

Example 60. The method of Example 58 or 59, wherein the at least one tooth comprises one or more of a central incisor, a lateral incisor, or a canine.

Example 61. The method of any one of Examples 55 to 60, wherein determining the amount of visible gingiva comprises measuring a distance between the lip contour and the tooth contour.

Example 62. The method of Example 61, wherein the distance is measured at a plurality of locations.

Example 63. The method of Example 61 or 62, wherein the distance is measured between the lip contour and a peak of the tooth contour corresponding to a gingival apex of an anterior tooth.

Example 64. The method of any one of Examples 55 to 63, wherein determining the amount of visible gingiva comprises measuring an area between the lip contour and the tooth contour.

Example 65. The method of any one of Examples 54 to 64, wherein determining the target arrangement comprises:
comparing the amount of visible gingiva to a threshold value, and
if the amount of visible gingiva exceeds the threshold value, adjusting a position of at least one tooth to reduce the amount of visible gingiva.

Example 66. The method of Example 65, wherein adjusting the position of the at least one tooth comprises intruding the at least one tooth.

Example 67. The method of Example 66, further comprising constraining an intrusion distance of the at least one tooth to be within a clinically acceptable tooth movement limit.

Example 68. The method of any one of Examples 54 to 67, wherein determining the target arrangement comprises:
generating a generic leveling surface,
generating an adjusted leveling surface based on the amount of visible gingiva in the 2D image,
comparing an amount of visible gingiva resulting from the generic leveling surface versus an amount of visible gingiva resulting from the adjusted leveling surface, and
selecting the generic leveling surface or the adjusted leveling surface for use in determining the target arrangement based on the comparison.

Example 69. The method of Example 68, wherein the generic leveling surface is generated without considering the amount of visible gingiva in the 2D image, and the adjusted leveling surface is configured to reduce the amount of visible gingiva in the 2D image.

Example 70. The method of Example 68 or 69, wherein:
the generic leveling surface is selected if the amount of visible gingiva resulting from the generic leveling surface is less than or equal to the amount of visible gingiva in the 2D image, and
the adjusted leveling surface is selected if the amount of visible gingiva resulting from the generic leveling surface is greater than the amount of visible gingiva in the 2D image.

Example 71. The method of any one of Examples 54 to 70, wherein the 2D image shows the patient's mouth in a smiling position.

Example 72. The method of any one of Examples 54 to 71, wherein the 2D image is a photograph.

Example 73. The method of any one of Examples 54 to 72, wherein the 2D image is received from a mobile device.

Example 74. The method of any one of Examples 54 to 73, further comprising generating one or more intermediate arrangements to reposition the patient's teeth toward the target arrangement.

Example 75. The method of any one of Examples 54 to 74, further comprising generating instructions for fabricating one or more dental appliances configured to reposition the patient's teeth toward the target arrangement.

Example 76. A system comprising:
a processor; and
a memory operably coupled to the processor and storing instructions that, when executed by the processor, cause the computing system to perform operations comprising:
   receiving a 2D image of a patient's face;
   determining an amount of visible gingiva in the 2D image; and
   determining a target arrangement for the patient's teeth based on the amount of visible gingiva.

Example 77. The system of Example 76, wherein the operations further comprise generating a lip contour and a tooth contour based on the 2D image, and the amount of visible gingiva is determined based on the lip contour and the tooth contour.

Example 78. The system of Example 77, wherein the lip contour corresponds to a border of a lip of the patient.

Example 79. The system of Example 78, wherein the lip contour corresponds to a lower border of an upper lip of the patient.

Example 80. The system of any one of Examples 77 to 79, wherein the tooth contour corresponds to a gingival margin of at least one tooth of the patient.

Example 81. The system of Example 80, wherein the at least one tooth comprises an anterior tooth of the patient's upper arch.

Example 82. The system of Example 80 or 81, wherein the at least one tooth comprises one or more of a central incisor, a lateral incisor, or a canine.

Example 83. The system of any one of Examples 77 to 82, wherein determining the amount of visible gingiva comprises measuring a distance between the lip contour and the tooth contour.

Example 84. The system of Example 83, wherein the distance is measured at a plurality of locations.

Example 85. The system of Example 83 or 84, wherein the distance is measured between the lip contour and a peak of the tooth contour corresponding to a gingival apex of an anterior tooth.

Example 86. The system of any one of Examples 77 to 85, wherein determining the amount of visible gingiva comprises measuring an area between the lip contour and the tooth contour.

Example 87. The system of any one of Examples 76 to 86, wherein determining the target arrangement comprises:
comparing the amount of visible gingiva to a threshold value, and
if the amount of visible gingiva exceeds the threshold value, adjusting a position of at least one tooth to reduce the amount of visible gingiva.

Example 88. The system of Example 87, wherein adjusting the position of the at least one tooth comprises intruding the at least one tooth.

Example 89. The system of Example 88, wherein the operations further comprise constraining an intrusion distance of the at least one tooth to be within a clinically acceptable tooth movement limit.

Example 90. The system of any one of Examples 76 to 89, wherein determining the target arrangement comprises:
generating a generic leveling surface,
generating an adjusted leveling surface based on the amount of visible gingiva in the 2D image,
comparing an amount of visible gingiva resulting from the generic leveling surface versus an amount of visible gingiva resulting from the adjusted leveling surface, and
selecting the generic leveling surface or the adjusted leveling surface based on the comparison.

Example 91. The system of Example 90, wherein the generic leveling surface is generated without considering the amount of visible gingiva in the 2D image, and the adjusted leveling surface is configured to reduce the amount of visible gingiva in the 2D image.

Example 92. The system of Example 90 or 91, wherein:
the generic leveling surface is selected if the amount of visible gingiva resulting from the generic leveling surface is less than or equal to the amount of visible gingiva in the 2D image, and
the adjusted leveling surface is selected if the amount of visible gingiva resulting from the generic leveling surface is greater than the amount of visible gingiva in the 2D image.

Example 93. The system of any one of Examples 76 to 92, wherein the 2D image shows the patient's mouth in a smiling position.

Example 94. The system of any one of Examples 76 to 93, wherein the 2D image is a photograph.

Example 95. The system of any one of Examples 76 to 94, wherein the 2D image is received from a mobile device.

Example 96. The system of any one of Examples 76 to 95, wherein the operations further comprise generating one or more intermediate arrangements to reposition the patient's teeth toward the target arrangement.

Example 97. The system of any one of Examples 76 to 96, wherein the operations further comprise generating instructions for fabricating one or more dental appliances configured to reposition the patient's teeth toward the target arrangement.

Example 98. A non-transitory computer-readable storage medium comprising instructions that, when executed by one or more processors of a computing system, cause the computing system to perform operations comprising:
receiving a 2D image of a patient's face;
determining an amount of visible gingiva in the 2D image; and
determining a target arrangement for the patient's teeth based on the amount of visible gingiva.

### CONCLUSION

Although many of the embodiments are described above with respect to systems, devices, and methods for orthodontic and/or restorative treatments, the technology is applicable to other applications and/or other approaches, such as other treatments applied to a patient's craniofacial region (e.g., orthognathic treatments, plastic surgery). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-15.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A system comprising:
a processor; and
a memory operably coupled to the processor and storing instructions that, when executed by the processor, cause the system to perform operations comprising:
receiving a 2D image of a patient's face;
generating at least one smile line based on the 2D image;
receiving a 3D digital representation of the patient's arch;
determining a correspondence between the 2D image and the 3D digital representation;
generating a 3D projection of the at least one smile line based on the correspondence; and
determining a target arrangement for the patient's teeth based on the 3D projection of the at least one smile line.

2. The system of claim 1, wherein the 3D projection of the at least one smile line comprises at least one smile plane.

3. The system of claim 2, wherein the at least one smile plane contains the at least one smile line and intersects a projection viewpoint.

4. The system of claim 3, wherein the projection viewpoint corresponds to a camera position that, when used to project the 3D digital representation onto a 2D plane, produces a 2D projection of the 3D digital representation that is similar to the 2D image.

5. The system of claim 1, wherein the 2D image and the at least one smile line are in a 2D reference frame, and the 3D digital representation and the 3D projection of the at least one smile line are in a 3D reference frame.

6. The system of claim 1, wherein the operations further comprise determining a current spatial relationship between at least one tooth of the 3D digital representation and the 3D projection of the at least one smile line.

7. The system of claim 6, wherein the operations further comprise comparing the current spatial relationship to a target spatial relationship between the at least one tooth and the 3D projection.

8. The system of claim 7, wherein:
the current spatial relationship comprises a current distance between the at least one tooth and the 3D projection, and
the target spatial relationship comprises a target distance between the at least one tooth and the 3D projection.

9. The system of claim 7, wherein the operations further comprise adjusting a position of the at least one tooth in the target arrangement if the current spatial relationship differs from the target spatial relationship.

10. The system of claim 6, wherein the at least one tooth comprises one or more of a central incisor, a lateral incisor, or a canine.

11. The system of claim 1, wherein the target arrangement is determined using an automated treatment planning algorithm.

12. The system of claim 11, wherein the automated treatment planning algorithm implements one or more rules for determining positions of the patient's teeth in the target arrangement, and wherein the one or more rules are based on the at least one smile line.

13. The system of claim 12, wherein the one or more rules define a penalty parameter based on a spatial relationship between at least one tooth and the 3D projection of the at least one smile line.

14. The system of claim 1, wherein determining the correspondence between the 2D image and the 3D digital representation comprises:
projecting the 3D digital representation onto a 2D plane, and
comparing the projection of the 3D digital representation with the 2D image.

15. The system of claim 1, wherein the 2D image shows the patient's mouth in a smiling position.

16. The system of claim 1, wherein the 2D image is a photograph.

17. The system of claim 1, wherein the 2D image is received from a mobile device.

18. The system of claim 1, wherein the at least one smile line comprises one or more of the following: a facial midline, an intercanine width line, a gingival line, an incisal edge line, a horizontal line, or a proportion line.

19. The system of claim 1, wherein the at least one smile line defines a target smile for the patient.

20. The system of claim 1, wherein the operations further comprise generating instructions for fabricating one or more dental appliances configured to reposition the patient's teeth toward the target arrangement.
